(19) United States
(12) Patent Application Publication
(10) Pub. No.: US 2024/0018093 A1
(43) Pub. Date: Jan. 18, 2024

(54) NEW AMMONIUM COMPOUNDS USEFUL AS SURFACTANTS

(71) Applicant: RHODIA OPERATIONS, Lyon (FR)

(72) Inventors: Olivier Back, Lyon (FR); Bala Naga Satyanarayana Thota, Singapore (SG)

(73) Assignee: RHODIA OPERATIONS, Lyon (FR)

(21) Appl. No.: 18/011,157

(22) PCT Filed: Mar. 29, 2021
(86) PCT No.: PCT/EP2021/058080

§ 371 Date: Dec. 16, 2022

Related U.S. Application Data
(60) Provisional application No. 63/128,985, filed on Dec. 22, 2020.

(30) Foreign Application Priority Data
Jun. 16, 2020 (WO) PCT/EP2020/066649

(51) Int. Cl. C07C 229/12; C09K 23/00

(52) U.S. Cl. CPC C07C 229/12; C09K 23/018

(57) ABSTRACT

The invention concerns new mono-ammonium compounds of formula (I) with surfactant properties and improved biodegradability. It concerns also new mixtures comprising such mono-ammonium compounds and di-ammonium compounds.

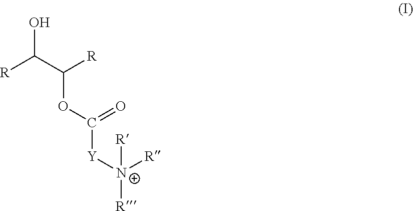

This application claims priorities filed on 16 Jun. 2020 as PCT patent application with Nr PCT/EP2020/066649 and filed on 22 Dec. 2020 in UNITED STATES with No. 63/128985, the whole content of each of these applications being incorporated herein by reference for all purposes.

The present invention relates to new ammonium compounds, in particular new quaternary ammonium compounds derivable from internal ketones, themselves obtainable from fatty acids or their derivatives and the use of the new ammonium compounds as surfactants, alone or in admixture with other surfactants.

Ammonium compounds which have surfactant properties and can be used in respective applications have been described in the literature and are available commercially in a variety of different types from various suppliers.

WO 97/08284 discloses compositions comprising Guerbet alcohol betaine esters which are represented by the formula

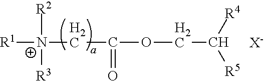

in which R¹ to R³ are independently selected from C₁ to C₄ alkyl groups or C₂-C₄ alkenyl groups, a is from 1 to 4 and R₄ and R₅ are independently selected from C₁₂ to C₂₂ alkyl or alkenyl groups, the sum of chain lenghts of R₄ and R₅ preferably being at least 30. Since the compounds are derived form Guerbet alcohols, the number of carbon atoms in groups R₄ and R₅ differs always by 2.

EP 721 936 is related to liquid quaternary ammonium compounds of the formula

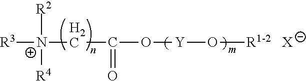

wherein R¹⁻² is a linear or branched C₃₆-C₄₄ alkyl or alkenyl group, R₂ to R₄ are C₁-C₅ alkyl or hydroxyalkyl groups, Y is a linear or branched C₂-C₄ alkylene group, m is a number of 0 to 20 and n is an integer of 1 to 6. Preferred compounds of EP 721 936 are, as in WO 97/08284, derived from Guerbet alcohols and are represented by the formula

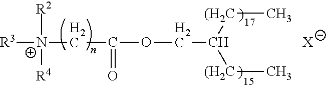

DE 3402146 relates to quaternary ammonium compounds. As in WO 97/08284 and EP 721 936, the compounds comprise two long chain substituents which are esters of Guerbet acids.

While fatty quaternary ammonium compounds are widely used as surfactants, there is still a need for compounds of this type having a good combination of surfactant properties on one hand and biodegradability on the other hand. Biodegradability has become more and more important in the recent past due to the desire of customers to have more environmentally friendly products. The improvement in biodegradability should not negatively affect the surfactant properties.

It was thus an object of the present invention to provide new ammonium compounds with good surfactant properties and a good biodegradability.

This object is achieved with the compounds of formula (I). Preferred embodiments of the present invention are also detailed hereinafter.

The novel ionic mono-ammonium compounds in accordance with the present invention have the formula (I) is

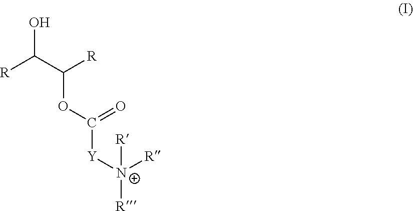

wherein R, which may be the same or different at each occurrence, is a C₅-C₂₇ aliphatic group, preferably a C₆ to C₂₄ aliphatic group,
Y is a divalent C₁-C₆ aliphatic group, and
R′, R″ and R″, which may be the same or different, are hydrogen or a C₁ to C₄ alkyl group.

The aliphatic groups R may be free of any double bond and of any triple bond. Alternatively, the aliphatic groups R may comprise at least one —C═C— double bond and/or at least one —C≡C— triple bond.

The aliphatic groups R are advantageously chosen from alkyl groups, alkenyl groups, alkanedienyl groups, alkanetrienyl groups and alkynyl groups.

The aliphatic groups R may be linear or branched.

Preferably, the aliphatic groups R are independently chosen from alkyl and alkenyl groups.

More preferably, the aliphatic groups R are independently chosen from alkyl and alkenyl groups, generally from C₆-C₂₄ alkyl and C₆-C₂₄ alkenyl groups, very often from C₆-C₂₁ alkyl and C₆-C₂₁ alkenyl groups and often from (i) C₆-C₁₉ alkyl and C₆-C₁₉ alkenyl groups or from (ii) C₆-C₁₇ alkyl and C₆-C₁₇ alkenyl groups. More preferably, R represent an alkyl group, generally a C₆-C₂₄ alkyl group, very often a C₆-C₂₁ alkyl group, often a C₆-C₁₉ alkyl group or a C₆-C₁₇ alkyl group. Aliphatic groups, in particular alkyl groups, with 10 to 20, preferably with 10 to 17 carbon atoms have been found much advantageous.

Acyclic aliphatic groups, more preferably linear aliphatic groups, still more preferably linear alkyl groups may be mentioned as preferred examples of substituents R. Excellent results were obtained when R was a linear alkyl group having from 14 to 17 carbon atoms.

The number of carbon atoms of R can be even or odd and each group R can have the same number of carbon atoms or the number of carbon atoms of different groups R may be different.

In some embodiments, either both R have an even number of carbon atoms or both R have an odd number of carbon atoms.

In some other embodiments, to which the preference is generally given for economic reasons, one and only one R has an odd number of carbon atoms and one and only one R has an even number of carbon atoms. In a particular embodiment which is advantageous from an economic standpoint, one and only one Rhas an odd number of carbon atoms n_{O} while the other R has an even number of carbon atoms n_{E}, wherein n_{E} is equal to n_{O}−1.

Let us represent the number of carbon atoms of the two R groups by couple (n^{1,} n²), wherein n¹ is the number of carbon atoms of the first R group and n² is the number of carbon atoms of the other R group. Exemplary and preferred couples (n^{1,} n²) are chosen from the following couples: (10, 11), (12, 13), (14, 15), (16, 17), (10, 13), (10, 15), (10, 17), (11, 12), (11,14), (11, 16), (12, 15), (12, 17), (13, 14), (13, 16), (14, 17) and (15, 16). Particularly preferred couples (n^{1,} n²) are chosen from the following couples: (14, 15), (16, 17),(14, 17) and (15, 16).

R′ is preferably a C₁ to C₄ alkyl group, preferably methyl or ethyl, more preferably methyl. Likewise, R″ is preferably a C₁ to C₄ alkyl group, preferably methyl or ethyl, more preferably methyl. Still likewise, R″′ is preferably a C₁ to C₄ alkyl group, preferably methyl or ethyl, more preferably methyl. Preferably at least one, more preferably at least two, more preferably all three of R′, R″ and R″′ are a C₁ to C₄ alkyl group, preferably methyl or ethyl, most preferably methyl.

Y is preferably an acyclic divalent C₁-C₆ aliphatic group, more preferably a linear divalent C₁-C₆ aliphatic group, still more preferably a linear alkanediyl (commonly referred to as “alkylene”) C₁-C₆ group. Besides, Y has preferably from 1 to 4 carbon atoms. Exemplary Y are ethanediyl and methanediyl (commonly referred to as “methylene”). Excellent results were obtained when Y was a methylene group.

In some embodiments, the ionic compound of formula (I) is chosen from ionic compounds C_{I}* wherein Y is methylene, R′, R″ and R″′ are methyl, and the two R groups are such that:
one R is n-tetradecyl while the other R is n-pentadecyl, or
one R is n-hexadecyl while the other R is n-heptadecyl, or
one R is n-pentadecyl while the other R is n-hexadecyl,
one R is n-tetradecyl while the other R is n-heptadecyl.

In some other embodiments, the ionic compound of formula (I) is chosen from compounds other than ionic compounds C_{I}*.

The present invention is also directed to electroneutral compounds of formula (II)

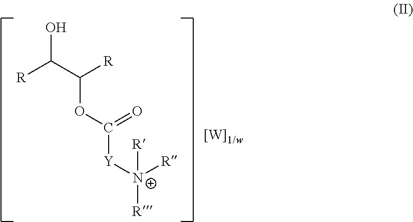

wherein R, R′, R″, R″′ and Y are as defined and described hereinbefore and W is an anion or an anionic group bearing w negative charges. Suitable anions or anionic groups W are e.g. halides such as chloride, fluoride, bromide or iodide, methyl sulfate or methosulfate anion (CH₃—OSO₃_{^{−}}), methanesulfonate anion (CH₃—SO₃_{^{−}}), sulfate anion, hydrogensulfate anion (HSO₄_{^{−}}) or an organic carboxylate anion such as acetate, propionate, benzoate, tartrate, citrate, lactate, maleate or succinate.

In some embodiments, the electroneutral compound of formula (II) is chosen from electroneutral compounds C_{II}* wherein [W]_{1/w} is chloride anion (Cl⁻, w being equal to 1), Y is methylene, R′, R″ and R′″ are methyl, and the two R groups are such that:
one R is n-tetradecyl while the other R is n-pentadecyl, or
one R is n-hexadecyl while the other R is n-heptadecyl, or
one R is n-pentadecyl while the other R is n-hexadecyl,
one R is n-tetradecyl while the other R is n-heptadecyl.

In some other embodiments, the electroneutral compound of formula (II) is chosen from electroneutral compounds other than compounds C_{II}*.

The compounds in accordance with the present invention can be obtained by a variety of processes. Preferred processes for the manufacture of the compounds of the present invention include the reaction of an internal ketone of formula R—C(═O)—R, which internal ketone may preferably be obtained by decarboxylative ketonization of a fatty acid, a fatty acid derivative or a mixture thereof. A suitable process for the manufacture of internal ketones following this route is diclosed in US 2018/0093936 to which reference is made for further details. Two processes for the synthesis of compounds of the present invention using internal ketones obtainable as indicated above as starting materials is now described.

The first process starts with a Piria ketonization followed by hydrogenation, dehydration, epoxidation (to obtain an epoxide) and epoxide ring opening reaction (to obtain a monohydroxyl-monoester). The epoxide ring opening reaction step is followed by an amine condensation step (as the final step) to convert the monoester into a compound complying with formula (I). This is a multi-step process plugged on Piria technology. It has the advantage of being salt-free and relying on chemical transformations which can be easily performed.

### First Process for Synthesis of Compounds of Formula (I)

### Piria Ketonization

The basic reaction in the first step is:

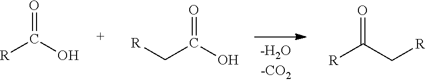

This reaction has been thoroughly described in U.S. Pat. No. 10,035,746, WO 2018/087179 and WO 2018/033607 to which reference is made for further details.

### Hydrogenation

The internal ketone is then subjected to hydrogenation which can be carried out under standard conditions known to the skilled person for hydrogenation reactions:

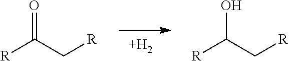

The hydrogenation reaction is conducted by contacting the internal ketone with hydrogen in an autoclave reactor at a temperature ranging from 15° C. to 300° C. and at a hydrogen pressure ranging from 1 bar to 100 bars. The reaction can be conducted in the presence of an optional solvent but the use of such solvent is not mandatory and the reaction can also be conducted without any added solvent. As examples of suitable solvents one can mention: methanol, ethanol, isopropanol, butanol, THF, methyl-THF, hydrocarbons, water or mixtures thereof. A suitable catalyst based on a transition metal should be employed for this reaction. As examples of suitable catalysts, one can mention heterogeneous transition metal based catalysts such as for example supported dispersed transition metal based catalysts or homogeneous organometallic complexes of transition metals. Examples of suitable transition metals are: Ni, Cu, Co, Fe, Pd, Rh, Ru, Pt, Ir. As examples of suitable catalysts one can mention Pd/C, Ru/C, Pd/Al2O₃, Pt/C, Pt/Al2O₃, Raney Nickel, Raney Cobalt etc. At the end of the reaction, the desired alcohol can be recovered after appropriate work-up. The skilled person is aware of representative techniques so no further details need to be given here. Details of this process step can e.g. be found in U.S. Pat. No. 10,035,746 to which reference is made here.

The skilled person will select suitable reaction conditions based on his professional experience and taking into account the specific target compound to be synthesized. Accordingly, no further details need to be given here.

### Dehydration

In the next step, the alcohol thus obtained is subjected to dehydration to obtain an internal olefin. This reaction can also be carried out under standard conditions known to the skilled person for respective dehydration reactions (e.g. U.S. Pat. No. 10,035,746, example 4) so that no further details need to be given here:

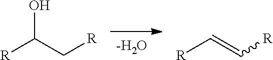

The dehydration reaction is conducted by heating the secondary alcohol in a reaction zone in the presence of a suitable catalyst at a temperature ranging between 100° C. and 400° C. The reaction can be conducted in the presence of an optional solvent but the use of such solvent is not mandatory and the reaction can also be conducted without any added solvent. As examples of solvents one can mention: hydrocarbons, toluene, xylene or their mixture. A catalyst must be employed for this reaction. Suitable examples of catalysts are acidic (Lewis or Bronsted) catalysts either heterogeneous solid acid catalysts or homogeneous catalysts. As examples of heterogeneous catalysts one can mention alumina (Al2O₃), silica (SiO₂), aluminosilicates (Al2O₃—SiO₂) such as zeolites, phosphoric acid supported on silica or alumina, acidic resins such as Amberlite® etc. Homogeneous catalysts can also be employed and one can mention the following suitable acids: H₂SO₄, HCl, trifluoromethanesulfonic acid, para-toluenesulfonic acid, AICl₃, FeCl₃ etc. Water that is generated during the reaction can be distilled out from the reaction medium in the course of the reaction. At the end of the reaction, the desired olefin can be recovered after appropriate work-up. The skilled person is aware of representative techniques and same are e.g. described in U.S. Pat. No. 10,035,746 so that no further details need to be given here.

As above indicated, in the ionic mono-ammonium compounds of formula (I), embodiments wherein one and only one R has an odd number of carbon atoms and one and only one R has an even number of carbon atoms are generally preferred for economic reasons. As it is now made apparent, this can happen when both R originate from a carboxylic acid having an even number of carbon atoms and is generally advantageous from an economic standpoint because fatty carboxylic acids of natural origin—which have typically such an even number of carbon atoms—are broadly available; this can also happen when both R originate from a carboxylic acid having an odd number of carbon atoms. In particular, embodiments wherein one and only one Rhas an odd number of carbon atoms n_{O} while the other R has an even number of carbon atoms n_{E} wherein n_{E} is equal to n_{O}−1, can occur when the internal olefin is obtained from one and only one carboxylic acid having an even number of carbon atoms. For illustration purposes, internal olefins of which couple (n^{1,} n²) representing the number of carbon atoms of the two R groups is chosen from (14, 15), (16, 17),(14, 17) and (15, 16) can be obtained starting from the following carboxylic acids or mixtures of carboxylic acids: palmitic acid alone, stearic acid alone, oleic acid alone, palmitic acid in admixture either with stearic acid or with oleic acid or with stearic acid and oleic acid, and stearic acid in admixture with oleic acid.

On the other hand, when one and only one R originates from a carboxylic acid having an even number of carbon atoms and one and only one R originates from a carboxylic acid having an odd number of carbon atoms, internal olefins and, at the end, ionic mono-ammonium compounds of formula (I) wherein either both R have an even number of carbon atoms or both R have an odd number of carbon atoms are obtained.

### Epoxidation

This internal olefin can thereafter be oxidized to the respective epoxide wherein the double bond is substituted by an epoxide group in accordance with the following scheme (where the reactants are just exemplary for respective groups of compounds serving the respective function):

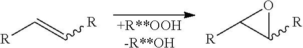

wherein R** can be hydrogen or a hydrocarbon group that can be substituted and/or interrupted by a heteroatom or heteroatom containing group, or R** can be an acyl group of formula R***—C(═O)—wherein R*** can have the same meaning as R**.

The epoxidation reaction is advantageously conducted by contacting the internal olefin with an appropriate oxidizing agent in a reaction zone at a temperature ranging usually from 15° C. to 250° C.

As suitable oxidizing agents one can mention peroxide compounds such as hydrogen peroxide (H₂O₂) that can be employed in the form of an aqueous solution, organic peroxides such as peracids of formula R****—CO₃H (for example meta-chloroperoxybenzoic acid, peracetic acid, etc.), hydrocarbyl (e.g. alkyl) hydroperoxides of formula R****′—O₂H (for example cyclohexyl hydroperoxide, cumene hydroperoxide, tert-butyl hydroperoxide) where R**** in the peracid or R****′ in the hydrocarbyl (e.g. alkyl) hydroperoxide is a hydrocarbon group (e.g. an alkyl group) that can be substituted and/or interrupted by a heteroatom or heteroatoms-containing group.

The reaction can be conducted in the presence of an optional solvent but the use of such solvent is not mandatory and the reaction can also be conducted without any added solvent. As example of suitable solvents one can mention: CHCl₃, CH₂Cl₂, tert-butanol or their mixtures.

When H₂O₂ is used as the oxidizing agent, the presence of an organic carboxylic acid during the reaction can be beneficial as it will generate in-situ a more reactive peracid compound by reaction with H₂O₂. As examples of suitable carboxylic acids one can mention: formic acid, acetic acid, propionic acid, butanoic acid, benzoic acid etc.

A catalyst can also be used to promote the reaction. Suitable catalysts are Lewis or Bronsted acids and one can mention for example: perchloric acid (HClO₄), trifluoromethanesulfonic acid, heterogeneous titanium silicalite (TiO₂—SiO₂), heterogeneous acidic resins such as Amberlite® resins, homogeneous organometallic complexes of manganese, titanium, vanadium, rhenium, tungsten, polyoxometellates etc.

At the end of the reaction, the desired epoxide can be recovered after appropriate work-up and the skilled person is aware of representative techniques so that no further details need to be given here.

The epoxide can be directly engaged in next step without further purification.

### Epoxide Ring Opening Reaction

The epoxide ring opening reaction can thereafter be achieved by reacting the epoxide with a carboxylic acid reagent to obtain a monohydroxyl-monoester compound of formula (III)

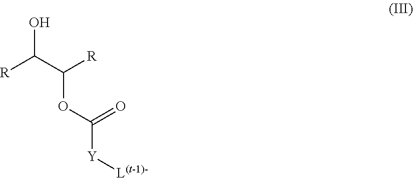

in accordance with the following scheme:

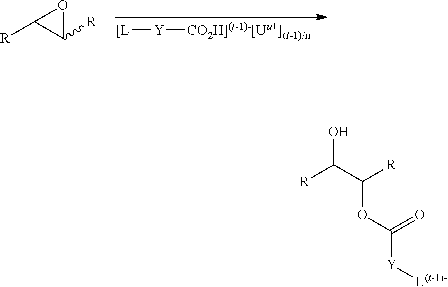

wherein, wherever present in the above compounds,
L is a leaving group,
t is an integer which is equal to 1 or which is equal or superior to 2,
U^{u+} is a cation,
u is an integer fixing the positive charge of the cation, and
R and Y are as previously described.

The epoxide ring opening reaction is performed by contacting the epoxide with a carboxylic acid reagent of formula (IV):

[L-Y—CO₂H]^{(t-1)-}[U^{u+}]_{(t-1)/u} (IV)

wherein L, Y, t, U^{u+} and u are as previously described.

The Applicant has surprisingly found that the epoxide can be directly converted into the monohydroxyl-monoester when using such a carboxylic acid reagent.

When t is equal to 1, no cation is present. Otherwise said, the epoxide ring opening reaction is performed by contacting the epoxide with a carboxylic acid of formula:

L-Y—CO₂H .

In the case the leaving group L already carries a negative charge in the carboxylic acid reagent (this is the case when (t−1) is equal or superior to 1, i.e. when t is equal or superior to 2), a cation noted U^{u+} (with u preferably being 1, 2 or 3, more preferably 1) must be present in the reactant to ensure the electroneutrality. This cation may e.g. be selected from H+, alkaline metal cations (e.g. Na⁺ or K⁺), alkaline earth metal cations (e.g. Ca²⁺), Al³⁺ and ammonium, to mention only a few examples.

The nature of the leaving group L is not particularly limited provided next reaction step (i.e. amine condensation, as will be detailed later on) can occur. The leaving group L is advantageously a nucleofuge group. It can be notably chosen from
a halogen,
a (hydrocarbyloxysulfonyl)oxy group of formula R^{a}—O—SO₂—O— wherein R^{a} denotes a C₁-C₂₀ hydrocarbyl group which can be optionally halogenated,
a (hydrocarbylsulfonyl)oxy group of formula R^{a}—SO₂—O— wherein R^{a} denotes a C₁-C₂₀ hydrocarbyl group which can be optionally halogenated (such as in CF3—SO₂—O—), and
an oxysulfonyloxy group of formula ⁻O—SO₂—O— (which is a leaving group L already carrying one negative charge on a terminal oxygen atom).

The hydrocarbyl group R^{a}, wherever present in here before formulae, can be notably an aliphatic group or an aromatic group such as phenyl or p-tolyl. The aliphatic group R^{a} is usually a C₁-C₆ alkyl group, which can be linear or ramified; it is often a linear C₁-C₄ alkyl, such as methyl, ethyl or n-propyl.

The leaving group L is preferably chosen from:
a halogen, such as fluorine, chlorine, bromine or iodine,
a (hydrocarbyloxysulfonyl)oxy group of formula R^{a}—O—SO₂—O— wherein R^{a} denotes a C₁-C₂₀ hydrocarbyl group, such as CH₃—O—SO₂—O—, and
an oxysulfonyloxy group of formula ⁻O—SO₂—O—.

An example for a compound with t equal to 1 is CH₃—O—SO₃—CH₂—COOH which can be designated as 2-((methoxysulfonyl)oxy)acetic acid. As further examples of compounds in which t is equal to 1 and thus no cation is present, one can mention: chloroacetic acid, bromoacetic acid and 2-chloropropionic acid.

An example for t being equal to 2 is sodium carboxymethylsulfate acid in which [L-Y—COOH]^{(t-1)-}[U^{u+}]_{(t-1)/u} is [O—SO₂—O—CH₂—COOH]⁻[Na⁺].

The reaction can be conducted in the presence of a solvent. However the presence of such solvent is not mandatory and the reaction can be also conducted without any added solvent. As example of suitable solvents one can mention: toluene, xylene, hydrocarbons, DMSO, Me-THF, THF or mixtures thereof.

The reaction is advantageously conducted under an inert atmosphere, such as a nitrogen or rare gas atmosphere. An argon atmosphere is an example of a suitable inert atmosphere.

The reaction can be conducted in the absence of any catalyst. A catalyst can also be employed during the reaction and suitable catalysts are Bronsted or Lewis acid catalysts. As preferred examples of catalysts one can mention: H₂SO₄, para-toluenesulfonic acid, trifluoromethanesulfonic acid, HCl, or heterogeneous acidic resins such as Amberlite® resins, AlCl₃, FeCl₃, SnCl₄, etc.

The total number of moles of the carboxylic acid reagent of formula (IV) which is contacted with the epoxide during the whole course of the reaction is advantageously no less than half of the total number of moles of epoxide; it is preferably at least as high as the total number of moles of epoxide, and it is more preferably at least twice higher than the total number of moles of epoxide. Besides, the total number of moles of carboxylic acid reagent which is contacted with the epoxide during the whole course of the reaction is advantageously at most ten times higher than the total number of moles of epoxide.

The reaction takes advantageously place in a reactor where the epoxide is in molten state. It has also been found advantageous that the reaction takes place in a reactor where the carboxylic acid reagent of formula (IV) is in molten state. Preferably, the reaction takes place in a reactor where both the epoxide and the carboxylic acid reagent are in molten state.

Advantageously, the epoxide is added progressively in a reactor containing the whole amount of of the carboxylic acid reagent formula (IV); preferably, it is added continuously in a reactor containing the whole amount of the carboxylic acid reagent, such as for example under a fed-batch process. The Applicant has observed that contacting progressively, preferably continuously, the epoxide with the whole amount of the carboxylic acid made it possible to limit the self condensation of the epoxide.

The epoxide ring opening reaction can be conducted at a temperature ranging generally from about 20° C. to about 200° C. in the presence of an optional solvent. To allow for a sufficient reaction rate, the reaction is preferably conducted at a temperature which is of at least 25° C., more preferably at least 45° C., still more preferably at least 55° C. On the other hand, the Applicant has surprisingly found that conducting the reaction at a high temperature resulted in the formation of a high amount of ketone, diester and dehydration by-products. Accordingly, the reaction is conducted at a temperature which is preferably below 120° C., more preferably below 100° C. and still more preferably of at most 85° C.

The temperature may be kept constant over the whole reaction. However, to achieve the best compromise between reaction rate (conversion) and selectivity in the monohydroxyl-monoester, the reaction temperature is preferably slightly increased over the course of the reaction, while remaining always within the ranges delimited by the above specified lower and upper limits, e.g. [45° C., 120° C.[, preferably [55° C., 85° C].

Accordingly, the reaction of present concern, whereby an epoxide ring of an epoxide is opened to obtain a monohydroxyl-monoester, is desirably conducted in accordance with a process which comprises:
a first step S₁ wherein the epoxide is reacted with a carboxylic acid reagent of formula (IV) at a temperature T₁ from 20° C. to 70° C. for a time t₁ which is sufficient to convert more than f₁=50 mol. % of the epoxide into the monohydroxyl-monoester;
a second step S₂ wherein unconverted epoxide and unconverted carboxylic acid reagent at step S₁ are reacted at a temperature T₂ above 70° C. but below 120° C. for a time t₂ which is sufficient to convert more than f₂=80 mol. % of the epoxide into the monohydroxyl-monoester.

Preferably, the whole amount of the epoxide is added progressively, or even better continuously, during part or all of step S₁, over a period of time t′1 representing at least 25%, preferably at least 40% of the total time t₁ of step S₁, in a reactor containing the whole amount of the carboxylic acid reagent of formula (IV).

T₁ is preferably of at least 35° C., more preferably at least 45° C., still more preferably at least 55° C. Good results were obtained when T₁ was about 65° C.

f₁ is preferably 70 mol. %.

t₁ ranges generally from 10 min to 10 h. t₁ is preferably of at least 30 min, more preferably of at least 1 h. Besides, t₁ is preferably of at most 4 h, more preferably of at most 2 h.

T₂ is preferably of at least 75° C. Besides, T₂ is preferably of at most 95° C., more preferably of most 85° C. Good results were obtained when T₂ was about 80° C.

f₂ is preferably 90 mol. %, more preferably 95 mol. %, still more preferably 98 mol. %.

t₂ ranges generally from 10 min to 10 h. t₂ is preferably of at least 30 min, more preferably of at least 1 h. t₂ is preferably of at most 4 h, more preferably of at most 2 h.

The whole reaction can be conducted at atmospheric pressure or at subatmospheric pressure. It is preferably conducted at atmospheric pressure or under a light vacuum, that is to say at a pressure from 90 kPa to the atomospheric pressure (about 1 atm=101.325 kPa). More preferably, it is conducted at atmospheric pressure.

Although the above operating conditions aim to a large extent at maximizing the amount of monohydroxyl-monoester and minimizing the amount of diester co-product of formula (V)

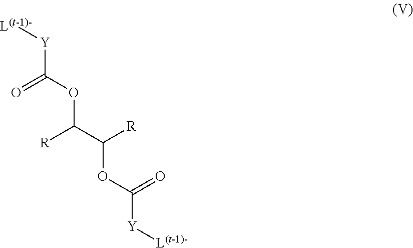

a certain amount of such a diester is however generally co-produced. The diester over (monohydroxyl-monoester+diester) molar ratio is generally below 50%, often of at most 30%, possibly of at most 15% or even at most 5% or 2%.

Other operating conditions may be applied which further restrain the manufacture of the diester compound, and allow a higher selectivity in the monohydroxyl-monoester compound. It can for example be mentioned:
(c1) having the total number of moles of carboxylic acid reagent of formula (IV) which is contacted with the epoxide during the whole course of the reaction equal of at most 1.10 times the total number of moles of epoxide, possibly from 0.10 to 1.00 time the total number of moles of epoxide or from 0.50 to 0.90 time the total number of moles of epoxide,
(c2) conducting the whole epoxide ring opening reaction of the epoxide with the carboxylic acid reagent of formula (IV) at a temperature T of at most 20° C. to 70° C., preferably at most 65° C., possibly at most 60° C. or at most 50° C.,
(c3) as the diester of formula (V) is formed through the consecutive esterification reaction of the monohydroxyl-monoester compound of formula (III) with the carboxylic acid reagent, the reaction progress can be interrupted, for example by cooling the reaction medium at a temperature at which the esterification reaction converting (III) to (V) does not evolve anymore (for example, at a temperature below 30° C.) or by removing the carboxylic acid reagent of formula (IV) (for example, through distillation under vaccum) or by neutralizing the carboxylic acid reagent by the addition of an at least equivalent amount of a base (for example, an aqueous NaOH solution), and
(c4) applying (c1) and (c2), or (c1) and (c3), or (c2) and (c3), or (c1), (c2) and (c3).

However, applying at least one of (c1), (c2) and (c3) has generally adverse effects on the productivity, reaction rate and/or yield in the monohydroxyl-monoester.

Further, as will be seen later on, the co-produced diester may result in the obtention of a di-ammonium compound which exhibits outstanding biodegradability and surfactant properties, as the mono-ammonium of formula (I) compound does, so that, in accordance with some embodiments of the present invention, it has been found advantageous to allow for the production of a certain amount of diester together with the monohydroxyl-monoester.

To favour the removal of water and the obtaining of diester, yet in an amount that is lower than the amount of monohydroxyl-monoester include, step S2 of the above detailed process can be partly or entirely conducted under vacuum, usually at a pressure P2 below 50 kPa, preferably of at most 30 kPa, more preferably at most 10 kPa, still more preferably at most 3 kPa, e.g. about 1 kPa. For example, step S₂ can be conducted in two parts, wherein temperature T₂ is firstly maintained at a pressure P21 from 90 kPa to the atmospheric pressure (about 1 atm=101.325 kPa), preferably at atmospheric pressure, then pressure P2 is decreased and maintained at a pressure P22 below 50 kPa, preferably of at most 30 kPa, more preferably at most 10 kPa, still more preferably at most 3 kPa. The decrease of the pressure P2 can be advantageously conducted with an increase in the temperature T2 during step S2: the second step S₂ can be conducted partly or entirely at a temperature T2 of at least 85° C. but below 120° C.; for example, step S2 can be conducted in two parts, wherein temperature T2 is firstly maintained at a temperature T21 from 70° C. but below 85° C., then temperature T2 can be increased and maintained at a temperature T22 of at least 85° C. but below 120° C. The first and second parts of step S2 relative to the increase of temperature T2 match preferably with, i.e. take preferably place during the same periods of time than, respectively the first and second parts of step S2 as defined for the decrease of pressure P2.

At the end of the reaction, the desired monohydroxyl-monoester compound of formula (III), optionally in combination with the diester compound of formula (V), can be recovered after appropriate work-up and the skilled person is aware of representative techniques so that no further details need to be given here.

### Amine Condensation

The monohydroxyl-monoester compound of formula (III) can be converted into the ionic mono-ammonium compound of formula (I) [or its electroneutral homologue of formula (II)] through the following reaction scheme:

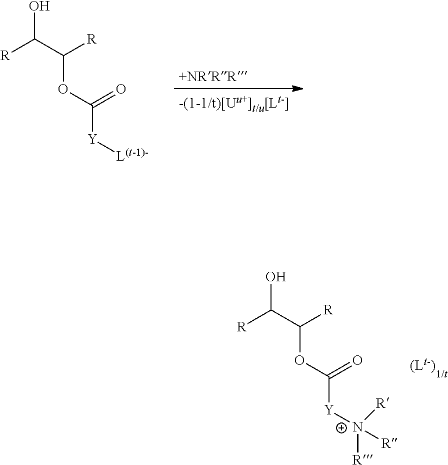

wherein R, R′, R″, R″′, Y, L, U, t and u are as described here before.

Optionally, together with the conversion of the monohydroxyl-monoester compound of formula (III) into the ionic mono-ammonium compound of formula (I) (or its electroneutral homologue), the diester compound of formula (V) can be converted into the di-ammonium compound of formula (VI)

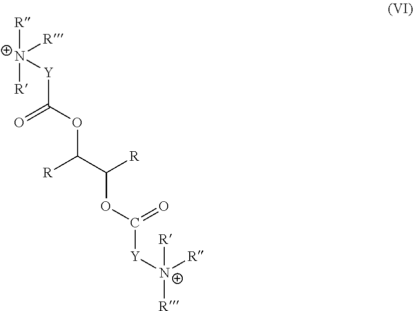

(or its electroneutral homologue) through the following reaction scheme:

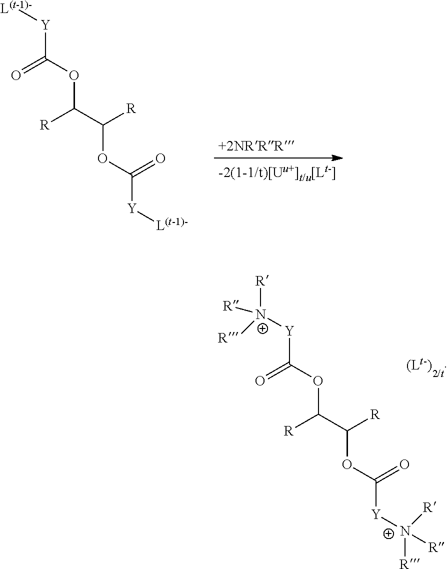

The amine condensation reaction is performed by contacting the intermediate monohydroxyl-monoester compound of formula (III), optionally together with the diester of formula (V), with ammonia or an amine of formula NR′R″R″′ where R′, R″ and R″′, which may be the same or different, are hydrogen or a C₁ to C₄ alkyl group, and preferred R′, R″ and R″′ are exactly as above defined in connection with the ionic mono-ammonium compound of formula (I).

The reaction can be conducted at a temperature ranging from 15° C.to 250° C. in the presence of a suitable solvent. As example of a suitable solvent one can mention: THF, Me-THF, methanol, ethanol, isopropanol, DMSO, toluene, xylene or their mixture. Alternatively the reaction can be also conducted in the absence of any added solvent.

During this reaction, there is a nucleophilic attack of ammonia or of the amine that substitutes L^{(t-1)-} in the monohydroxyl-monoester or the diester; L^{(t-1)-} plays the role of the leaving group. L^{t-} becomes then the counter-anion of the final ammonium compound. In the case the leaving group already carries a negative charge in the monohydroxyl-monoester or diester reagent (this is the case when (t−1) is equal or superior to 1 or when t is equal or superior to 2) there is also formation of a salt as the by-product of the reaction with the general chemical formula [U^{u+}]_{t/u}[L^{t-}].

### Other Process for Synthesis of Compounds of Formula (I)

### Acyloin Condensation

An alternative process for the synthesis of compounds of formula (I) proceeds via an acyloin condensation in accordance with the following scheme:

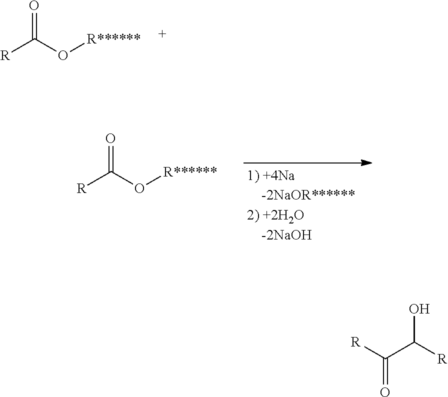

wherein R****** is an alkyl group having from 1 to 6 carbon atoms.

The acyloin condensation is generally performed by reacting an ester (typically a fatty acid methyl ester) with sodium metal as the reducing agent. The reaction be performed in a high boiling point aromatic solvent such as toluene or xylene where the metal can be dispersed at a temperature above its melting point (around 98° C. in the case of sodium). The reaction can be conducted at a temperature ranging from 100° C. to 200° C. At the end of the reduction, the reaction medium can be carefully quenched with water and the organic phase containing the desired acyloin product can be separated. The final product can be obtained after a proper work-up and the skilled person is aware of representative techniques so that no further details need to be given here.

Reactions of this type have been described in the literature, e.g. in Hansley, J. Am. Chem. Soc 1935, 57, 2303-2305 or van Heyningen, J. Am. Chem. Soc. 1952, 74, 4861-4864 or in Rongacli et al., Eur. J. Lipd Sci. Technol. 2008, 110, 846-852 , to which reference is made herewith for further details.

### Keto-Alcohol Hydrogenation

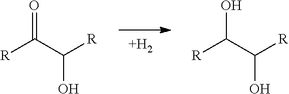

This reaction can be conducted using the conditions described hereinbefore for the first process variant for the manufacture of compounds of formula (I).

The obtained diol can then be directly esterified with the carboxylic acid reagent of formula (IV) according to a classical Fisher esterification reaction. Standard conditions to perform esterification reactions are well known in the art so that no further details need to be further given here. During the course of the reaction, as there are two alcohol functions that can be esterified, there is first formation of the hydroxy-monoester (III) which can then be converted into the bis-ester (V) in a consecutive reaction. The ratio between monoester (III) and diester (IV) can be controled during this step by limiting the conversion of (III) to (V) according to the methods (c1) and/or (c3) that are given in paragraph [0083].

Finally the mixture of esters (III) and (IV) are converted to the corresponding ammonium compounds (I) and (VI) respectively according to the conditions described previously for the quaternization reaction.

The exemplary processes described before are examples of suitable processes, i.e. there might be other suitable processes to synthesize the compounds in accordance with the present invention. The processes described hereinbefore are thus not limiting as far as the methods of manufacture of the compounds according to the present invention is concerned.

The compounds of formula (I) (and their electroneutral homologues) can be used as surfactants. Surfactants are compounds that lower the surface tension (or interfacial tension) between two liquids, a liquid and a gas or between a liquid and a solid. Surfactants may act as detergents, wetting agents, emulsifiers, foaming agents, and dispersants.

Surfactants are usually organic compounds that are amphiphilic, meaning they contain both hydrophobic groups (their tails) and hydrophilic groups (their heads). Therefore, a surfactant contains both a water-insoluble (or oil-soluble) component and a water-soluble component. Surfactants shall diffuse in water and adsorb at interfaces between air and water or at the interface between oil and water, in the case where water is mixed with oil. The water-insoluble hydrophobic group may extend out of the bulk water phase, into the air or into the oil phase, while the water-soluble head group remains in the water phase.

The adsorption of a cationic surfactant on negatively charged surfaces is an important property for such surfactants. This property is usually linked to the minimum concentration of surfactant needed to produce aggregation of a negatively charged cellulose nanocrystal (CNC, which is often used as reference material) suspension in aqueous media. Consecutive variation of size can be monitored and followed by dynamic light scattering (DLS).

Following the protocol described in E. K. Oikonomou et al., J. Phys. Chem. B, 2017, 121 (10), 2299-307 the adsorption properties of ammonium compounds can be investigated by monitoring the ratio X=[surfactant]/[CNC] or the mass fraction M=[surfactant]/([surfactant+[CNC]), at fixed [surfactant]+[CNC]=0.01 wt % in aqueous solution, required to induce the agglomeration of the cellulose nanocrystals.

The biodegradability of the compounds of the present invention can be determined in accordance with procedures described in the prior art and known to the skilled person. Details about one such method, OECD standard 301, are given in the experimental section hereinafter.

The compound of formula (I) (or its electroneutral homologue) exhibits outstanding surfactant properties and biodegradability.

It can be used in various aqueous or hydro-alcoholic formulations as the sole ammonium compound exhibiting surfactant properties, i.e. no other mono-ammonium compound exhibiting surfactant properties and no di- or higher ammonium compound exhibiting surfactant properties are present in these formulations.

The Applicant has observed that, in aqueous or hydro-alcoholic formulations, the compounds of formula (I) structured generally in the form of lamellae, such as multilamellar vesicles. This lamellar structure resulted generally in aqueous or hydro-alcoholic formulations exhibiting a substantially higher viscosity than the same formulations but based on an ammonium surfactant which structures in the form of micelles. This higher viscosity is well adapted to some applications, while for some other applications a somewhat lower viscosity is desired.

On the other hand, many di-ammonium compounds are structured in the form of micelles, resulting in aqueous or hydro-alcoholic formulations exhibiting a lower viscosity. This lower viscosity is well adapted to certain applications, while for other applications a higher viscosity is desired, which can be either similar to the one achieved with the compounds of formula (I) or intermediate between the viscosities achieved with the compounds of formula (I) on the one hand and di-ammonium compounds on the other hand.

There is a need for materials which, further to exhibiting outstanding surfactant properties and fairly good to excellent biodegradibilility, are capable of forming in aqueous or hydro-alcoholic formulations in a broad range of viscosities, so as to mach with the various viscosity requirements as required by various end use applications.

This meet is satisfied with a mixture M_{Q} comprising:
at least one ionic mono-ammonium compound of formula (I) as above described, and
at least one ionic di-ammonium compound of formula (VII)

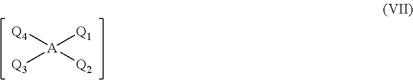

wherein
A is a tetravalent linker selected from the group consisting of A-1 to A-6

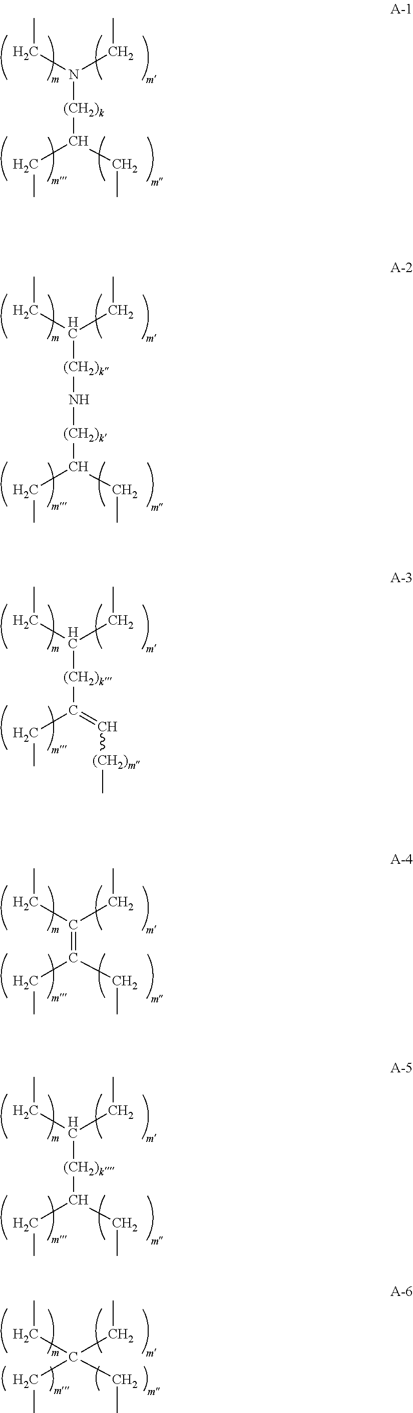

m, m′, m″ and m″′, which may be the same or different at each occurrence, are 0, 1, 2 or 3,
k, k′ k″, k″′ and k″″, which may be the same or different, are 0, 1, 2 or 3, Q₁ to Q₄, which may be identical or different from each other, are selected from the group consisting of R and X,
R, which may be the same or different at each occurrence, is as previously defined for the compound (I),
X, which may be the same or different at each occurrence, is represented by formula (VIII)

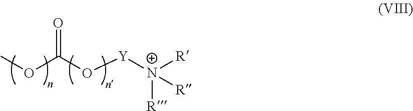

wherein
two and only two of Q₁ to Q₄ are represented by X and two and only two of groups Q₁ to Q₄ are represented by R,
Y, which may be the same or different at each occurrence, is as previously defined for the compound (I),
R′, R″ and R″′, which may be the same or different at each occurrence, are as previously defined for the compound (I), and
n and n′, which may be the same or different at each occurrence, are 0 or 1 with the sum of n+n' being 1 or 2.

This need is also satisfied with a mixture M′_{Q} comprising:
at least one electroneutral compound of formula (II) as above described, (i.e. an electroneutral homologue of the compound of formula (I) as above described), and
at least one electroneutral compound of formula of formula (IX)

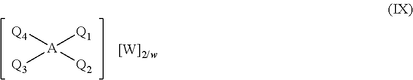

(i.e. an electroneutral homologue of the compound of formula (VII) as above described),
wherein A and Q₁ to Q₄, which may be identical or different from each other, are as above described for the compound of formula (VII), and W is an anion or an anionic group bearing w negative charges.

Examples of suitable anions or anionic groups W are as above specified for the electroneutral compound of formula (II).

The Applicant has found that the di-ammonium compounds of formula (VII), like the mono-ammonium compounds of formula (I), exhibit outstanding surfactant properties.

The Applicant has also found that the di-ammonium compounds of formula (VII) exhibit a fairly good to excellent biodegradability, with an emphasis for the compounds of formula (VI) which, like the compounds of formula (I), exhibit excellent biodegradability.

The Applicant has finally found that the di-ammonium compounds of formula (VII), notably the compounds of formula (VI), are structured in the form of micelles, and can form aqueous or hydro-alcoholic formulation exhibiting a lower viscosity than the compounds of formula (I).

By adjusting the respective amounts of the compound of formula (I) and the compound of formula (VII) in the mixture M_{Q} (or of their electroneutral homologues in the mixture M′_{Q}), aqueous or hydro-alcoholic formulations in a broad range of viscosities can be prepared, complying with the various viscosity requirements as required by various end use applications.

Preferably, the compound of formula (VII) is selected from the group consisting of compounds of formulae (VI), (X), (XI), (XII) and (XIII), as represented here below:

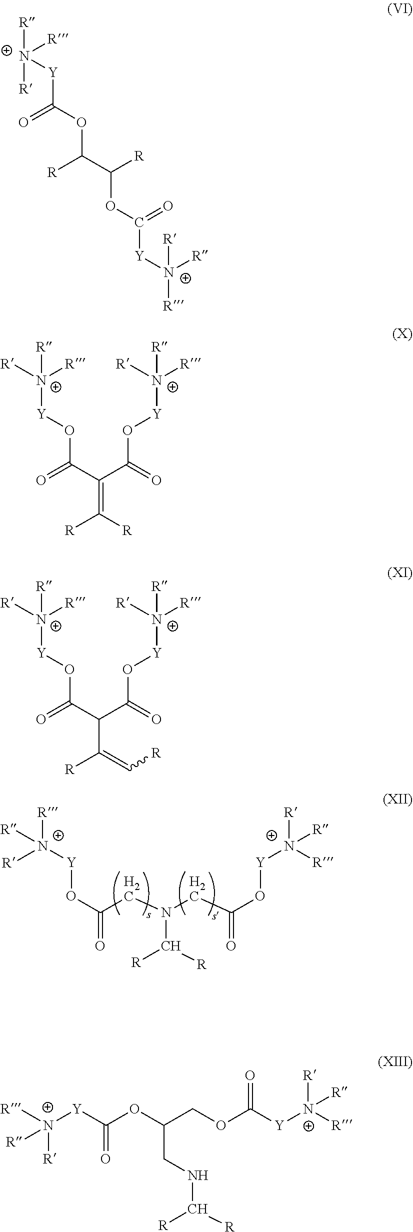

wherein
R, R′, R″, R″′ and Y, which may be the same or different at each occurrence, are as above described for the compound (I), and s and s′, which may be the same or different, are 0, 1, 2 or 3.

Preferably, the compound of formula (VII) is a compound of formula (VI).

The ratio w_{I, VII} of the weight of the compound (I) over the combined weight of the compound (I) and the compound (VII) in the mixture M_{Q} may vary to a large extent, depending on the applications where M_{Q} is intended to be used. The ratio w_{I, VII} ranges generally from 1% to 99%, very often from 10% to 90%. It may be of at least 20%, at least 30%, at least 40%, at least 50%, at least 60%, at least 70% or at least 80%. Besides, it may be of at most 80%, at most 70%, at most 60%, at most 50%, at most 40%, at most 30% or at most 20%. Examples of suitable ranges are [20%, 90%], [30%, 90%], [40%, 90%], [50%, 90%], [60%, 90%], [20%, 80%], [30%, 80%], [40%, 80%], [50%, 80%], [60%, 80%], [20%, 70%], [30%, 70%], [40%, 70%], [50%, 70%] and [60%, 70%]. These examplified ranges may notably be well adapted for various applications using mixtures M_{Q} wherein the compound of formula (VII) is a compound of formula (VI).

Likewise, the ratio w_{II, IX} of the weight of the compound (II) over the combined weight of the compound (II) and the compound (IX) in the mixture M′_{Q} a may vary to a large extent, depending on the applications where M′_{Q} is intended to be used. The ratio w_{II, IX} ranges generally from 1% to 99%, very often from 10% to 90%. It may be of at least 20%, at least 30%, at least 40%, at least 50%, at least 60%, at least 70% or at least 80%. Besides, it may be of at most 80%, at most 70%, at most 60%, at most 50%, at most 40%, at most 30% or at most 20%. Examples of suitable ranges are [20%, 90%], [30%, 90%], [40%, 90%], [50%, 90%], [60%, 90%], [20%, 80%], [30%, 80%], [40%, 80%], [50%, 80%], [60%, 80%], [20%, 70%], [30%, 70%], [40%, 70%], [50%, 70%] and [60%, 70%]. These examplified ranges may notably be well adapted for various applications using mixtures M′_{Q} wherein the compound of formula (IX) is an electroneutral homologue of the compound of formula (VI).

Some non-optimized mixtures M′_{Q} have been described in examples 11 and 12 of PCT/EP2020/066649. The mixture of example 11 comprises a quaternary mono-ammonium compound and a diquaternary ammonium compound in a weight ratio w_{II/IX} of about 2/97, while the mixture of example 12 comprises the same compounds in a weight ratio w_{II/IX} of about 5/92; in both examples, the combined weight of the quaternary mono-ammonium compound and the diquaternary ammonium compound constitutes about 97% of the total weight of the mixture. The quaternary mono-ammonium compounds of examples 11 and 12 are electroneutral compounds C_{II}* as above described. In general, the mixture M′_{Q} differs from a mixture similar to the mixtures of examples 11 and 12, that is to say that, in general, M′_{Q} differs from a mixture comprising at least one electroneutral compound of formula (II) chosen compounds C_{II}* and at least one electroneutral compound of formula (IX), wherein the electroneutral compound of formula (II) and the electroneutral compound of formula (IX) are in a weight ratio w_{II/IX} of about 2/97 (meaning typically, from 2/96 to 2/98) or about 5/92 (meaning typically from 5/9 1to 5/93) and the combined weight of the electroneutral compound of formula (II) and the electroneutral compound of formula (IX) constitutes about 97% (meaning typically from 96% to 98%) of the total weight of the mixture M′_{Q}.

The mixtures M_{Q} and M′_{Q} may comprise respectively the ionic compound of formula (I) and the ionic compound of formula (VII), or the electroneutral compound of formula (II) and the electroneutral compound of formula (IX) in a combined weight amount of at least at least 0.1%, at least 0.2%, at least 0.5%, at least 1%, at least 2%, at least 5%, at least 10%, at least 20%, at least 50% or at least 90%. The mixture M′_{Q} may consist essentially of the electroneutral compound of formula (II) and the electroneutral compound of formula (IX). In addition to the ionic compound of formula (I) and the ionic compound of formula (VII), the mixture M_{Q} may comprise water or water plus an alcohol such as ethanol, propanol or butanol. The mixture M_{Q} may consist essentially of (i) the ionic compound of formula (I), (ii) the ionic compound of formula (VII), and (iii) water or water in combination with an alcohol such as ethanol, propanol or butanol.

All along this description as well as in the below working examples, any developed formula has to be understood as involving, if appropriate, all potential enantiomers and diastereoisomers. No specific stereochemistry is targeted, in the absence of specific mention, each presented chiral molecule is in the form of its racemic mixture.

Should the disclosure of any patents, patent applications, and publications which are incorporated herein by reference conflict with the description of the present application to the extent that it may render a term unclear, the present description shall take precedence.

### WORKING EXAMPLES

### Example 1 —Synthesis of a Quaternary Mono-Ammonium Compound of Formula (I) Starting from C₁₆-C₁₈ (30:70) Fatty Acid Cut

Part 1.A—Piria Ketonization Toward Internal C₃₁-C₃₅ Ketones Cut

The reaction was conducted under an inert argon atmosphere in a 200 mL quartz reactor equipped with a mechanical stirring (A320-type stirring mobile manufactured by 3D-printing with Inox SS₃₁₆ L), an insulated addition funnel, a distillation apparatus, a heating mattress and a temperature probe.

In the reactor were introduced:
12.5 g of MASCID™ acid 1865 (from Musim Mas Group) composed of 33.7 wt % of palmitic acid and 65.3 wt % of stearic acid (0.045 mole of fatty acids), and
0.935 g of MgO (0.023 mole).

In the insulated addition funnel were added 37.5 g of the same melted fatty acids mixture (0.135 mole).

The temperature of the reaction media was then raised to 250° C. Once the temperature reached 150° C., stirring was started (1200 rpm). After 2 h00 reaction time at 250° C., FTIR analysis showed complete conversion of the starting fatty acids into the intermediate magnesium carboxylate complex.

The temperature of the reaction mass was then raised further to 330° C. and the mixture was allowed to stir at this temperature during 1 h 30 in order to allow decomposition of the intermediate magnesium carboxylate complex to the desired ketone.

Then, 12.5 g of the melted fatty acid mixture was progressively added into the reactor thanks to the addition funnel during 30 minutes and the mixture was stirred at 330° C. during an additional 1 h 00. FTIR analysis showed complete conversion of fatty acids and magnesium complex to the desired ketone.

Two additional cycles of 12.5 g fatty acid addition during 30 minutes followed by additional 1 h 00 stirring at 330° C. were then realized.

After the last cycle the mixture was allowed to stir at 330° C. during an additional 1 h 00 to ensure complete conversion of the intermediate magnesium complex to the desired ketone which was confirmed by FTIR analysis.

The temperature of the reaction mixture was then allowed to cool down at room temperature and the crude was solubilized in hot CHCl₃. The suspension was filtered on a plug of silica (70 g) and the product was further eluted with additional amounts of CHCl₃.

After solvent evaporation 41.83 g (0.086 mole) of product was obtained as a white wax corresponding to an isolated yield of 96%.

¹H NMR (CDCl₃, 400 MHZ) δ (ppm): 2.45-2.25 (t, J=7.6 Hz, 4H), 1.62-1.46 (m, 4 H), 1.45-1.05 (m, 54 H), 0.86 (t, J=6.8 Hz , 6H).

¹³C NMR (CDCl3, 101 MHZ) δ (ppm): 212.00, 43.05, 32.16, 29.93, 29.91, 29.88, 29.84, 29.72, 29.65, 29.59, 29.51, 24.13, 22.92, 14.34 (terminal CH₃).

Part 1.B—Hydrogenation of Ketones Mixture Toward Internal C₃₁-C₃₅ Fatty Alcohols Mixture

In a 100 mL autoclave equipped with a mechanical stirrer (Rushton turbine) were added:
4.36 g of Ru/C (4.87% Ru) catalyst (5 wt % of dry catalyst with respect to the ketone, catalyst containing 54.9% H2₂O)
39.3 g (87.2 mmol) of melted internal C₃₁-C₃₅ ketones cut.

The reaction was performed under 20 bar hydrogen pressure. 4 nitrogen purges are performed followed by 3 purges of hydrogen at 20 bars. The temperature of the reaction mixture was then set at 100° C. to melt the ketone substrate. The temperature was left at 100° C. during 10 min and stirring was slowly started at 200 rpm. When proper stirring was confirmed, the stirring rate was increased at 1200 rpm and the temperature was set at 150° C.

After 6 h reaction time at 150° C., heating was stopped and the mixture was allowed to cool down at 90° C. while stirring. Stirring was then stopped. The mixture was cooled down to room temperature and the autoclave was carefully depressurized.

NMR analysis in CDCl₃ of the crude showed a ketone conversion level >99% and molar purity of 99% for the fatty alcohol. The compact solid containing the product and the catalyst was grounded to powder and then introduced into a 1 L flask. 500 mL of chloroform were added and the flask was then heated at 60° C. to dissolve completely the alcohol. The suspension was filtered at 60° C. over celite. The solid cake was rinsed with hot chloroform at 60° C. several times. The filtrate was evaporated to give white powder with a weight purity of about 99% for the desired internal C₃₁-C₃₅ fatty alcohols mixture corresponding to about 90% isolated yield.

Part 1.C—Dehydration of C₃₁-C₃₅ Fatty Alcohols Into Internal Olefins

All the reactions were conducted under an inert argon atmosphere.

In a 200 mL quartz reactor equipped with a heating mattress, a mechanical stirrer (A320-type stirring mobile manufactured by 3D-printing with Inox SS₃₁₆ L), surmounted by a condenser connected to a 50 mL two-neck distillate collection flask and a temperature probe were added:
41.3 g of C₃₁-C₃₅ fatty alcohols (85 mmol, 1 eq.), and
4.13 g (40 mmol, 10 wt %) of Al2O₃-η.

The temperature of the reaction media was increased to 150° C. to melt the alcohol and stirring was started (about 500 rpm). Then, the temperature was set-up at 300° C. and the mixture was allowed to stir at 1000 rpm under argon. The reaction progress was monitored thanks to NMR analysis with a borosilicate glass tube.

After 2 hours reaction at 300° C., NMR analysis in CDCl₃ showed complete conversion of the fatty alcohol and the presence of 1.5 mol % of ketone which had been formed as a by-product.

Stirring and heating were then stopped and the temperature was lowered to 80° C. The molten crude was transferred to a beaker. The reactor vessel and the stirring mobile were rinsed with chloroform (Al2O₃ is insoluble).

The mixture was filtered and the solvent was evaporated under vacuum to afford 39 g of a clear yellow oil which solidified at room temperature to give a white solid in the form of wax (98 wt % purity) corresponding to 97% yield (NMR).

¹H NMR (CDCl₃, 400 MHZ) δ (ppm): 5.38-5.29 (m, 2H), 2.03-1.93 (m, 4H), 1.35-1.19 (m, 55H (average H number)), 0.86 (t, J=6.8 Hz , 6H).

¹³C NMR (CDCl3, 101 MHz) δ (ppm): 130.6, 130.13, 32.84, 32.16, 30.01, 29.93, 29.8, 29.6, 29.55, 29.4, 22.93, 14.35 (terminal CH₃).

### Part 1.D—Epoxidation of Internal Olefins to Afford C₃₁₋₃₅ Oxiranes

The reaction was conducted under an inert argon atmosphere.

In a 300 mL double-jacketed reactor equipped with a mechanical stirrer (propeller with four inclined plows) and baffles, a condenser and a temperature probe were added:
38.2 g of C₃₁₋₃₅ internal olefins (98 wt % purity, 80 mmol)
6.9 mL (7.2 g, 120 mmol) of acetic acid, and
11.3 g (30 wt %) of Amberlite® IR 120H resin.

The mixture was heated to 75° C. to melt the fatty alkene. The agitation was then started and 12.3 mL (13.7 g, 120 mmol) of H₂O₂ 30% were slowly added into the mixture using an addition funnel while monitoring temperature of the reaction medium to prevent temperature increase of the reaction mass (exothermicity). This required about 20 min. During the addition, the agitation was increased to improve transfers due to the heterogeneous nature of the reaction media.

At the end of the addition, the temperature of the reaction medium was increased at 85° C. and after 6 h 10 of stirring at this temperature, NMR analysis showed that the conversion level was around 99% with 98% selectivity.

Heating was then stopped and 150 mL of chloroform were added when the temperature of the reaction mass was around 50° C. The mixture was transferred to a separating funnel and the organic phase was washed 3 times with 150 ml of water. The resin catalyst that stayed in the aqueous phase was removed during phase separation. The aqueous phase was extracted twice with 50 mL of chloroform. The organic phase was dried over MgSO₄, filtered and evaporated to afford 39.2 g of a white solid with a purity of 98 wt % (epoxide+dialcohol by-product). The yield taking into account the purity was 99%.

¹H NMR (CDCl₃, 400 MHZ) δ (ppm): 2.91-2.85 (m, 1.5H), 2.65-2.6 (m, 0.5H), 1.53-1.36 (m, 4H), 1.35-1.19 (m, 55H (aver. H number)), 0.86 (t, J=6.8 Hz, 6H).

¹³C NMR (CDCl₃, 101 MHZ) δ (ppm): 58.97, 57.28, 32.18, 31.96, 29.72, 29.6, 29.4, 27.86, 26.95, 26.63, 26.09, 22.72, 14.15 (terminal CH₃).

### Part 1.E—Epoxide Ring Opening With Chloroacetic Acid to Afford Chloroacetate Monoester C₃₁₋₃₅

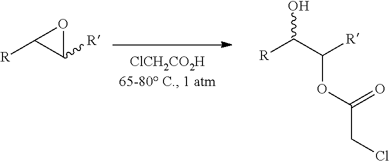

The reaction was conducted under an inert argon atmosphere. In a 500 ml three necked round bottom flask equipped with a magnetic stirrer, a heater, a condenser, a temperature probe and an insulated addition funnel, were added 44.2 g of chloroacetic acid (463 mmol, 5 eq.)

In the insulated addition funnel maintained at 80° C. were added 45 g of melted C₃₁-35 fatty epoxide (purity: 99.97 wt % , 92.6 mmol, 1 eq.)

The 1st step of hydroxy-ester formation through oxirane opening was conducted at 65° C. to limit the formation of ketone and dehydration by-products. The melted fatty epoxide was progressively added drop-wise over 1 h 20 into the reaction media containing melted chloroacetic acid under stirring at 65° C. The progressive addition of epoxide was carried out in order to limit by-products formed by condensation between two epoxide molecules. At the end of epoxide addition, the mixture was stirred at 65° C. during 1 h 30.

The 2nd step of hydroxy-ester formation through oxirane opening was conducted by an additional stirring at 80° C. during 1 h 00.

NMR analysis (CDCl₃) of the crude showed complete conversion of the starting epoxide and a 88:12 mol % monoester : bisester mixture composition.

### Part 1.F—Optional Further Reaction With Chloroacetic Acid to Afford the Partial Conversion of the Chloroacetate Monoester C₃₁-35 Into the Corresponding Diester

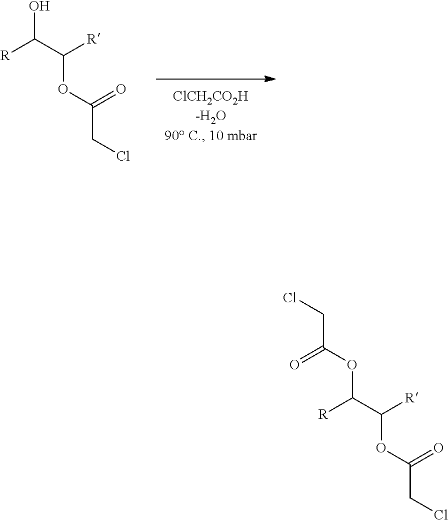

The condenser was replaced by a curved distillation column and the temperature of the reaction medium, that is to say the previously obtained crude having a 88:12 mol % monoester:bisester mixture composition, was increased to 90° C. followed by a progressive pressure decrease down to 10 mbar in order to distillate chloroacetic acid excess and to remove water formed as by-product.

After 1 h 30 distillation at 90° C. (10 mbar), ¹H NMR analysis showed a monoester : bisester ratio of 74:26 mol % with remaining chloroacetic acid.

At this stage the distillation was stopped and the mixture was allowed to cool down to room temperature. The crude was then solubilized into 150 ml of toluene and transferred into a separating funnel. The organic phase was washed 3 times with 150 ml of an aqueous NaOH solution (0.1M) followed by 150 ml of brine. The organic phase was separated, dried over MgSO₄, filtered and evaporated to give 53 g of a residual beige oil.

¹H NMR (CDCl₃) after solvent evaporation showed the approximate composition of the beige oil: 66 wt % (70 mol %) of chloroacetate hydroxy-ester, 26 wt % (25 mol %) of chloroacetate bisester, 5 wt % (3 mol %) of monoester dimer, 2 wt % (1 mol %) of bisester dimer, 0.2 wt % (0.3 mol %) of ketone and 0.2 wt % (1 mol %) of chloroacetic acid.

The final yield in the chloroacetate mono+bisester taking into account the purity of the mixture was ˜88%.

¹H NMR (CDCl₃, 400 MHz) δ (ppm): 5.11-5.02 (m, 2H, diester), 4.96-4.83 (m, 1H, monoester), 4.07 (s, 1H, monoester), 4.06 (s, 1H, monoester), 4.04 (s, 2H, diester), 4.03 (s, 2H, diester), 3.74-3.67 (m, 1H, isomer 1, monoester), 3.64-3.54 (m, 1H, isomer 2, monoester), 1.73-1.61 (m, 2H, monoester), 1.61-1.48 (m, 4H, diester), 1.48-1.36 (m, 2H, monoester), 1.36-1.12 (m, 55 H (average number)), 0.86 (t, J=6.8 Hz , 6H).

¹³C NMR (CDCl₃, 101 MHZ) δ (ppm): 167.39, 167.27, 167.15, 167, 79.84, 78.97, 76.21, 75.83, 72.95, 72.41, 41.06, 41.01, 40.90, 40.80, 33.63, 32.18, 31.98, 30.57, 29.75, 29.72, 29.65, 29.59, 29.5, 29.42, 28.85, 28.61, 25.9 25.6, 24.48 25.33, 24.97, 22.74, 14.15 (terminal CH₃).

### Part 1.G—Quaternization With NMe₃

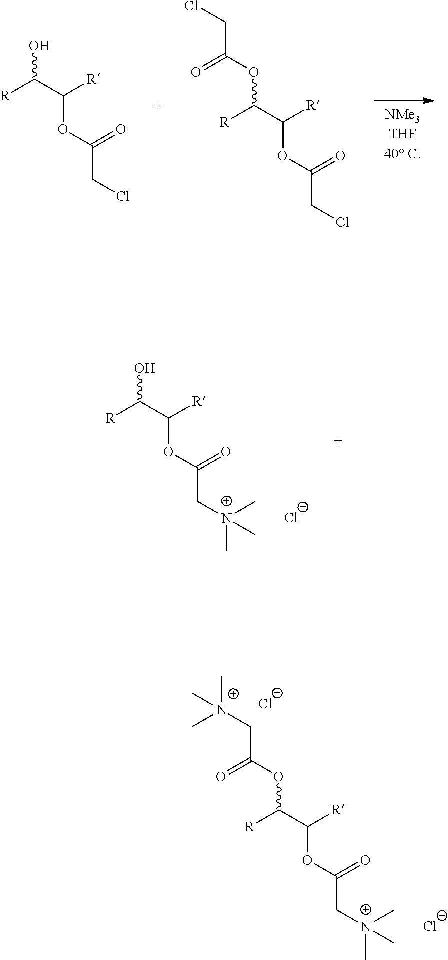

The reaction was conducted under an inert argon atmosphere. In a double-jacketed 1 L reactor equipped with a mechanical stirrer, a condenser, a temperature probe, a trap containing 0.1N HCl solution followed by a second trap containing activated carbon pellets, were added:
52 g (92.4 wt % purity, 80 mmol, 1 eq.) of a mixture of about 72 wt. % (74 mol. %) chloroacetate hydroxy-ester and about 28 wt.% (26 mol. %) of chloroacetate bisester, as obtainable upon completion of part 1-F, and
171 ml (320 mmol, 4 eq.) of a trimethylamine/THF solution (13 wt % concentration).

The reaction mixture was then heated at 40° C. and stirred at 1000 rpm. Reaction progress was followed up thanks to ¹H NMR analysis. After 6 h 00 stirring at 40° C., NMR analysis (CD3OD) showed complete conversion of chloroacetate esters and selective formation of the corresponding glycine betaine esters with the following approximate composition: 70 mol % of glycine betaine hydroxy-ester and 25 mol % of glycine betaine bisester.

The reactor was drained, rinsed with THF and the solvent was evaporated under vacuum to afford 58.8 g of a beige wax with the following weight composition: 65.2 wt % glycine betaine monohydroxy-ester, 27.6 wt % of glycine betaine bisester, 4.7 wt % of dimer monoester, 2.2 wt % of dimer bisester and 0.18 wt % of ketone.

The global yield in glycine betaine monohydroxy-ester plus glycine betaine bisester taking into account product purity was 98%. The glycine betaine monohydroxy-ester over (glycine betaine monohydroxy-ester plus glycine betaine bisester) weight ratio was 70%.

¹H NMR (MeOD-d4, 400 MHZ) δ (ppm): 5.17-5.06 (m, 2H, diquat), 5.02-4.87 (m, 1H, monoquat), 5.26-4.17/4.84-4.76/4.6-4.51/4.47-3.32 (m, 2H:monoquat, 4H:diquat), 3.41 (s, 18H, isomer 1, diquat), 3.38 (s, 18H, isomer 2, diquat), 3.36 (s, 9H, monoquat), 3.72-3.64 (m, 1H, isomer 1, monoquat), 3.56-3.47 (m, 1H, isomer 2, monoquat), 1.75-1.53 (m, 2H, monoquat), 1.53-1.44 (m, 4H, diquat), 1.44-1.35 (m, 2H, monoquat), 1.35-1.12 (m, 55 H (average number)), 0.86 (t, J=6.8 Hz , 6H).

¹³C NMR (MeOD-d4, 101 MHZ) δ (ppm): 165.46, 165.17, 81.33, 80.77, 77.17, 76.46, 72.35, 72.18, 63.89, 63.81, 63.54, 63.08, 54.46, 54.37, 54.22, 33.70, 32.51, 32.06, 31.18, 30.27, 30.03, 29.94, 29.8, 29.04, 28.8, 26.6, 26.3, 26.1, 26, 25.8, 23.24, 14.45 (terminal CH₃).

### Part 1.H—Purification of a Crude Richer in Chloroacetate Monoester C₃₁₋₃₅

A crude having a 88:12 mol % monoester:bisester mixture composition as obtainable upon completion of part 1-E is allowed to cool down to room temperature. The crude is then solubilized into toluene and transferred into a separating funnel. The organic phase is washed 3 times with an aqueous NaOH solution (0.1M) followed by brine. The organic phase is separated, dried over MgSO₄, filtered and evaporated to give a purified material rich in chloroacetate monoester C₃₁₋₃₅, having approximately a 88:12 mol % monoester:bisester mixture composition, and an overall monoester plus bisester content of about 95 wt. %.

Part 1.I—Quaternization With NMe₃ of a Crude Richer in Chloroacetate Monoester C₃₁₋₃₅

A quaternization reaction of the purified material obtained upon completion of part 1.H is achieved using the same quaternization reaction and purification protocols as described under part 1.G.

At the end, a purified surfactant material QA₂ having approximately a 90:10 wt. % glycine betaine monohydroxy-ester: glycine betaine bisester mixture composition, and an overall glycine betaine bisester plus glycine betaine monoester content of about 95 wt. %, is obtained.

### Example 2—Determination of Biodegradability

Biodegradability of test substances is measured according to the 301 F OECD protocol.

A measured volume of inoculated mineral medium, containing a known concentration of a test substance in order to reach about 50 to 100 mg ThOD/l (Theoretical Oxygen Demand) as the nominal sole source of organic carbon, is stirred in a closed flask (Oxitop™ respirometric flask) at a constant temperature (20+2° C.) for up to 28 days. Oxitop™ respirometric bottles are used in this test in order to access the biodegradability of the test sample: sealed culture BOD flasks were used at a temperature of 20+2 ° C. during 28 days.

Evolved carbon dioxide is absorbed by pellets of Natrium or Potassium hydroxide present in the head space of the bottle. The amount of oxygen taken up by the microbial population (=oxygen consumption expressed in mg/l) during biodegradation process (biological oxidation of the test substance) decreases the pressure of the head space (ΔP measured by the pressure switch) and is mathematically converted in mg O₂ consumed/litre. Inoculum corresponds to a municipal activated sludge washed in mineral medium (ZW media) in order to decrease the DOC (Dissolved Oxygen Carbon) content. Control solutions containing the reference substance sodium acetate and also toxicity control (test substance+reference substance) are used for validation purposes. Reference substance, sodium acetate, is tested in one bottle (at a nominal concentration of 129 mg/l corresponding to 100 mg ThOD/l) in order to check the viability of the inoculum. Toxicity control corresponds to the mixture of the substance reference and the test substance; it will check if the test substance is toxic towards the inoculum (if so, the test has to be redone at a lower test substance concentration, if feasible regarding the sensitivity of the method).

As the compounds and mixtures of compounds of the present invention are usually poorly soluble in water (and for those which are soluble in water, their metabolite after hydrolysis containing the alkyl chain has often very low solubility in water), we use a specific protocol named the “emulsion protocol”. This protocol enables us to increase the bioavailability of the poorly water-soluble substances in the aqueous phase where we have the inoculum.

Emulsion protocol consists of adding the test substance in the bottle through a stock solution made in an emulsion.

Emulsion is a 50/50 v/v mixture of a stock solution of the test substance dissolved in a non-biodegradable surfactant (Synperonic® PE 105 at 1 g/l) and then mixed with a mineral silicone oil AR 20 (Sigma).

The first dissolution of the test substance in the non-biodegradable surfactant solution often requires magnetic stirrer agitation followed by ultrasonication.

Once the dissolution is made, we mix the aqueous solution with a mineral silicone oil at a 50/50 volume/volume ratio. This emulsion is maintained by magnetic stirrer agitation and is sampled for an addition in the corresponding bottle in order to reach the required test substance concentration.

Two emulsion controls are run in parallel during the test in order to remove their value from the emulsion bottle containing the test substance added through the emulsion stock solution.

Biodegradability tests are achieved on the 70/30 w/w and 90/10 w/w glycine betaine monohydroxy-ester/glycine betaine bisester mixtures QA₁ and QA₂ of example 1. After 28 days, biodegradability is at least about 60% (OECD 301F). Similarly to the glycine betaine bisester taken alone, as reported herein after in Table 4, the compounds QA₁ and QA₂ displays final biodegradability rates over 60% after 28 days.

Thus, the glycine betaine monohydroxy-ester and the glycine betaine bisester contained in the mixture of example 1 exhibit outstanding biodegradability. This beneficial effect is achieved without detrimentally affecting the surfactant properties of the compounds.

### Example 3—Evaluation of Adsorption Properties on Nanocellulose Crystals

Adsorption of cationic surfactant on negatively charged surface is an important property for various applications. This property is linked to the minimal concentration of cationic surfactant needed to produce aggregation of negatively charged cellulose nano crystal (CNC) in suspension in aqueous media. Comparison of the aggregate size can be monitored by dynamic light scattering (DLS).

Following the protocol described in literature (Ref.: E. K. Oikonomou, et al., J. Phys. Chem. B, 2017, 121 (10), pp 2299-2307), adsorption properties of ammonium compounds are investigated by monitoring the ratio X=[surfactant]/[CNC] or the mass fraction M=[surfactant]/([surfactant]+[CNC]), at fixed [surfactant]+[CNC]=0.01wt % in aqueous solution, required to induce the agglomeration of the cellulose nano crystal.

The range of CNC aggregation corresponds to the range of ratio X (or M) triggering an aggregation of CNC, i.e. the range where the aggregate size measured by DLS is higher than a pure aqueous solution of CNC or an aqueous solution of surfactant at 0.01wt %.

Ranges of X and M of aggregation of CNC are summarized in Table 1 for 70/30 w/w and 90/10 w/w glycine betaine monohydroxy-ester/glycine betaine bisester mixtures QA₁ and QA₂ of example 1. Fentacare® TEP is used as a comparison. Fentacare® TEP is a commercial surfactant representing the benchmark.

The lower range of aggregation X or M, the better the adsorption properties on negatively charged surface.

TABLE 1

| | Range of CNC | Range of CNC |
|---|---|---|
| | aggregation (Ratio) | aggregation |
| | X = [surfactant]/[CNC] | (Mass fraction) |
| Cationic surfactant | Xₘᵢₙ − Xₘₐₓ | Mₘᵢₙ − Mₘₐₓ |
| Fentacare ® TEP | 1-33 | 0.50-0.97 |
| 70/30 w/w glycine | <<Xₘᵢₙ − Xₘₐₓ range of | <<Mₘᵢₙ − Mₘₐₓ range |
| betaine monohydroxy- | Fentacare ® TEP | of Fentacare ® TEP |
| ester/glycine betaine | | |
| bisester mixture QA₁ | | |
| of example 1 | | |
| 90/10 w/w glycine | <<Xₘᵢₙ − Xₘₐₓ range of | <<Mₘᵢₙ − Mₘₐₓ range |
| betaine monohydroxy- | Fentacare ® TEP | of Fentacare ® TEP |
| ester/glycine betaine | | |
| bisester mixture QA₂ | | |
| of example 1 | | |

The data show that the surfactant properties of the mixture of compounds of formulae (I) and (VI) in accordance with the present invention is superior compared to the commercial surfactant Fentacare® TEP.

So are also the surfactant properties of the compounds of formulae (I) and (VI) taken individually. The surfactant properties of the compounds of formulae (I) and (VI) and of mixtures thereof are further similar to the properties of the mixture of compounds of formulae (X) and (XI) as synthesized under Example 4—Part B for which values are reported in Table 5.

### Example 4—Additional Mixtures of Monoquaternary Ammonium Compounds of Formula (I) With Diquatemary Ammonium Compounds

Part 4.A—Synthesis of a diquaternary ammonium compound of formula (VI) starting from C₃₁ 16-hentriacontanone

### a) Obtention of C₃₁ Internal Olefin

C₃₁ internal olefin was obtained from palmitic acid according to the protocol described in U.S. Pat. No. 10,035,746, example 4.

### b) Epoxidation of Internal Olefin to Fatty Epoxide

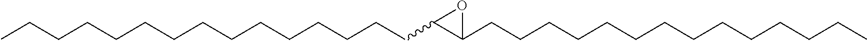 [SMILES: CCCCCCCCCCCCCCCC1OC1CCCCCCCCCCCCCC]

The reaction was conducted under an inert argon atmosphere.

In a 1 L double-jacketed reactor equipped with a mechanical stirrer (propeller with four inclined plows), a condenser, an addition funnel and a temperature probe were added 61.9 g of C₃₁ alkene (0.142 mol), followed by 16.3 mL (17.1 g, 0.285 mol) of acetic acid and 13.6 g (22 wt %) of Amberlite® IR 120H resin. The mixture was heated to 65 ° C.to melt the fatty alkene. The agitation was started and then 21.8 mL (24.2 g, 0.214 mol) of an aqueous solution of H₂O₂ (conc. 30%) was slowly added to the mixture using the addition funnel at a rate avoiding a significant temperature increase. This required about one hour. The temperature was then increased to 75° C. and the reaction mixture was allowed to stir overnight (after 15 min, NMR analysis showed that the conversion level was already around 60% with 99% selectivity). Then additional 10.2 mL (11.3 g, 0.1 mol) of an aqueous solution of H₂O₂ (30%) was added slowly and after 4 hours following the second addition of H₂O₂ NMR analysis showed that the conversion level was around 88% (98% selectivity). Another addition of 8.14 mL of acetic acid (8.55 g, 0.142 mol) followed by 11.6 mL of 30% H₂O₂ (12.91 g, 0.114 mol) was finally performed in order to increase the conversion level.

The mixture was allowed to stir a second night at 75° C.

Finally NMR analysis showed a conversion level of 93% (95% selectivity).

The mixture was allowed to cool down to room temperature and then 300 mL of chloroform were added. The mixture was transferred to a separating funnel and the organic phase was washed three times with 300 ml of water and then the aqueous phase was extracted twice with 100 mL of chloroform. The Amberlite® solid catalyst stayed in the aqueous phase and was removed during the first separation with the aqueous phase. The organic phases were collected, dried over MgSO₄, filtered and evaporated to give 65.3 g of a white solid with a purity of 91% w/w (epoxide+dialcohol).

The yield taking into account the purity was 92%.

¹H NMR (CDCl₃, 400 MHZ) δ (ppm): 2.91-2.85 (m, 2H, diastereoisomer 1), 2.65-2.6 (m, 2H, diastereoisomer 2), 1.53-1.00 (m, 54H), 0.86 (t, J=6.8 Hz, 6H).

¹³C NMR (CDCl₃, 101 MHZ) δ (ppm): 58.97, 57.28, 32.18, 31.96, 29.72, 29.6, 29.4, 27.86, 26.95, 26.63, 26.09, 22.72, 14.15 (terminal CH₃).

### c) Hydrolysis of Fatty Epoxide to Afford Fatty Diol

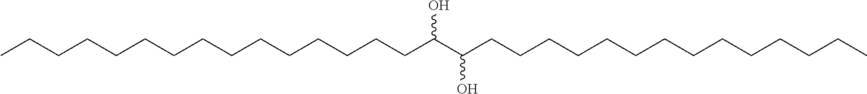 [SMILES: CCCCCCCCCCCCCCCC(O)C(O)CCCCCCCCCCCCCC]

The reaction was conducted under an inert argon atmosphere.

In a 1 L double-jacketed reactor equipped with a mechanical stirrer (propeller with four inclined plows), a condenser and a temperature probe were added 82.9 g of C₃₁ epoxide (purity: 94.5 wt %, 0.174 mol) followed by 480 ml of methyl-THF.

The mixture was allowed to stir at room temperature and 73 mL of a 3 M aqueous solution of H₂SO₄ was then added. The reaction medium was then stirred at 80° C. during 90 minutes. NMR analysis showed that the reaction was completed. The biphasic mixture was allowed to cool down to room temperature and the organic phase was separated. The solvent was then removed under vacuum and the residue was suspended in 200 mL of diethyl ether. The suspension was filtered and the resulting solid was washed 3 times with 50 mL of diethyl ether. The white solid was finally washed 2 times with 50 ml of methanol and was dried under vacuum to remove traces of solvent.

At the end 75.53 g of product was obtained as a white powder with a purity of 95.7% w/w corresponding to a yield of 89%.

¹H NMR (CDCl₃, 400 MHz) δ (ppm): 3.61-3.55 (m, 2H, diastereoisomer 1), 3.43-3.25 (m, 2H, diastereoisomer 2), 1.88 (brd, J=2.4 Hz, OH, diastereoisomer 2), 1.72 (brd, J=3.2 Hz, OH, diastereoisomer 1), 1.53-1.10 (m, 54H), 0.86 (t, J=6.8 Hz , 6H).

¹³C NMR (CDCl₃, 101 MHZ) δ (ppm): 74.71, 74.57, 33.66, 31.96, 31.23, 29.71, 29.39, 26.04, 25.68, 22.72, 14.15 (terminal CH₃)

### d) Esterification of Fatty Diol With Trimethylglycine to Afford Compound of Formula (VI)

All the reactions were conducted in carefully dried vessels and under an inert argon atmosphere.

Fresh commercial anhydrous CHCl₃ (amylene stabilized) and anhydrous toluene were used as such.

Betaine hydrochloride (19.66 g, 128.4 mmoles) was washed ten times with 20 mL of anhydrous THF followed by drying under vacuum to remove traces of solvent prior to use.

In a 100 mL four-neck round-bottom flask equipped with a magnetic stirrer, a heater, a condenser, a temperature probe and a curved distillation column connected to two traps of NaOH were quickly added:
19.66 g of dried betaine hydrochloride (128.4 mmoles) and
28 mL of SOCl₂ (45.86 g, 0.386 mol).

The heterogeneous mixture was stirred and the temperature was then slowly increased to 70° C. It was observed that when the temperature reached 68° C., gas was released (SO₂ and HCl) and the mixture turned homogeneous yellow.

The mixture was then allowed to stir at 70° C. during two hours and hot anhydrous toluene (25 mL, 80° C.) was added into the vessel. The mixture was stirred and then decanted at 0° C. (white-yellow precipitate formation) and the upper phase of toluene was removed through a cannula. The operation of toluene washing was repeated seven times in order to remove all SOCl₂ excess. NMR analysis showed complete conversion of glycine betaine hydrochloride but also formation of NMe₃·HCl adduct (NMe₃·HCl content in the solid: 12.3 mol %).

20 mL of dry CHCl₃ was then added to the solid betainyl chloride.

A solution of 26.19 g (56 mmol) of fatty diol in 90 mL of anhydrous CHCl₃ was prepared at 55° C. and was added dropwise under stirring to the reaction vessel at room temperature (exothermicity and emission of HCl was observed). The mixture was then allowed to stir at 55° C. overnight. Over the course of the reaction, the mixture turned homogeneously orange. NMR analysis showed that the conversion level was around 100%.

The mixture was then allowed to cool down to room temperature and the solvent was evaporated under vacuum.

The residue was solubilized in methanol at 0° C. and the formed precipitate was filtered out. The obtained filtrate was then evaporated to give 39.7 g of crude product.

This product was then deposited on a sinter filter and washed with cyclohexane to remove some remaining organic impurities. The resulting washed solid was dried under vacuum to afford 22 g of crude material. A final purification with a mixture of CH₂Cl_{2/}cyclohexane 50:50 was carried out; the solid was solubilized again in this solvent mixture at 50° C. and was allowed to cool down to room temperature. The formed precipitate was filtered out and after evaporation of the filtrate 19 g of a beige wax QA₃ was obtained with the following composition:
95 wt % of glycine betaine diester, corresponding to a compound of formula (VI)
1.5 wt % of methyl betainate
2 wt % of trimethylamine hydrochloride
1.5 wt % of glycine betaine hydrochloride.

The purified yield was 44%. No presence of glycine betaine monoester compound of formula (I) was identified in wax QA₃.

¹H NMR (MeOD-d4, 400 MHZ) δ (ppm): 5.3-5.2 (m, 2H), 4.68 (d, J=16.8 Hz, 2H), 4.50 (d, J=16.8 Hz, 2H), 4.53 (s, 1H), 4.48 (s, 1H), 3.37 (s, 18H), 1.75-1.55 (m, 4H), 1.39-1.10 (m, 50 H), 0.9 (t, J=6.8 Hz , 6H).

¹³C NMR (MeOD-d4, 101 MHZ) δ (ppm): 164.58, 75.76, 62.43, 53.10, 31.68, 30.05, 29.41, 29.38, 29.33, 29.28, 29.15, 29.09, 28.96, 24.71, 22.34, 13.05 (terminal CH₃).

### Part 4.B—Synthesis of a Mixture of Diquaternary Ammonium Compounds of Formulae (X) and (XI) Starting From C₃₁-16-Hentriacontanone

### a) Knoevenagel Condensation to Afford Diester Intermediate:

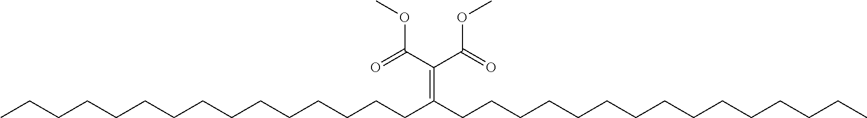 [SMILES: CCCCCCCCCCCCCCCC(CCCCCCCCCCCCCCC)=C(C(=O)OC)C(=O)OC]

All the reactions were conducted in carefully dried vessels and under an inert argon atmosphere.

Fresh commercial anhydrous CHCl₃, anhydrous THF and anhydrous pyridine were used as such.

In a 1 L double-jacketed reactor equipped with a mechanical stirrer (propeller with four inclined plows), a condenser, an addition funnel and a temperature probe were added 36.5 mL of TiCl₄ (63.00 g, 0.332 mole), followed by 146.3 mL of CHCl₃.

The mixture was stirred at −10° C. and anhydrous THF (358 mL) was slowly added through the addition funnel at a rate avoiding a temperature increase of the reaction medium above +5° C. During THE addition, a yellow precipitate appeared. Then 15.3 mL of dimethyl malonate (17.69 g, 0.134 mole) were added into the reaction mixture which was then allowed to stir at room temperature for 1 hour in order to allow malonate complexation to occur.

Then the mixture was allowed to cool down to 0° C. and a solution of 71.80 mL of anhydrous pyridine (70.50 g, 0.891 mole) in 23 mL of THF was slowly added into the reactor. During addition, the colour of the mixture turned red. The mixture was then allowed to stir at room temperature during 20 minutes to allow deprotonation to occur.

Finally, 50.00 g of C₃₁ ketone (0.111 mole) was added into the reaction mixture which was allowed to stir at room temperature during one night and during one more day at 35° C. 250 ml of water were then carefully added into the reactor followed by 250 mL of diethyl ether. The organic phase was separated and washed 4 times with 250 ml of water and one time with 200 mL of a saturated aqueous NaCI solution in order to remove pyridinium salts. The aqueous phases were combined and re-extracted with 3 times 250 mL of diethyl ether. The final organic phase was dried over MgSO₄, filtered and evaporated under vacuum to afford 70.08 g of crude orange oil. At this stage the crude contains residual amount of starting ketone as well as a main impurity corresponding to the condensation (aldolisation+crotonisation) of 2 equivalents of ketone.

The product could be easily purified by dissolving the oil in ethanol (the by-product and the starting ketone being not soluble in ethanol) followed by a filtration over celite.

The filtrate was evaporated, re-dissolved in CHCl₃, filtered again and evaporated to afford 52.57 g of oil with 95% of purity (RMN).

The overall purified yield was 79%.

¹H NMR (CDCl₃, 400 MHZ) δ (ppm): 3.68 (s, 6H), 2.32-2.19 (m, 4H), 1.45-1.39 (m, 4H), 1.30-1.10 (m, 48 H), 0.81 (t, J=6.4 Hz , 6H).

¹³C NMR (CDCl₃, 101 MHZ) δ (ppm): 166.30, 164.47, 123.65, 52.15, 34.61, 32.15, 30.16, 29.92, 29.91, 29.87, 29.76, 29.60, 28.65, 22.92, 14.34 (terminal CH₃).

### b) Transesterification With Dimethylaminoethanol to Afford Diamine Mixtures Intermediates:

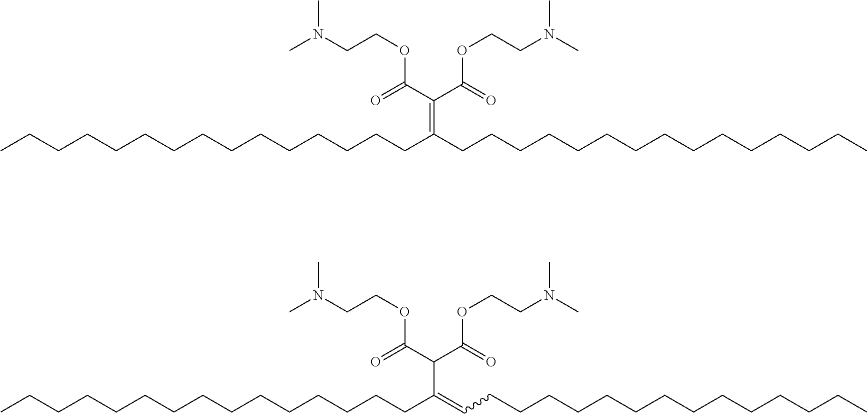

All the reactions were conducted in carefully dried vessels and under an inert argon atmosphere.

Fresh commercial anhydrous toluene and dimethylaminoethanol were used as such.

In a 2 L double-jacketed reactor equipped with a mechanical stirrer (propeller with four inclined plows), a condenser with a distillation apparatus and a temperature probe were added 42.7 g of the internal ketone/dimethyl malonate adduct (75.6 mmol) followed by 50 ml of toluene. The mixture was stirred at room temperature and 30.4 mL of dimethylaminoethanol (26.9 g, 302.2 mmol, 4 eq.) was added to the reaction system followed by 50 mL of toluene. Then 0.9 g of the catalyst dibutyltin oxide (3.8 mmol, 5 mol %) was added to the reaction mixture followed by 200 ml of toluene.

Then the mixture was allowed to stir at 120° C. and the reaction progress was followed by NMR analysis. To run a proper analysis an aliquot of the reaction medium was sampled and diluted in diethyl ether, quenched with water, decanted and the organic phase was evaporated under vacuum to be analysed in CDCl₃ NMR solvent. After 4 days of stirring at 120° C. NMR analysis showed that the conversion level was around 83% with 91% selectivity. In addition, by-product methanol was also present in the distillation flask. The reaction mixture was then allowed to cool down at room temperature and quenched with 500 mL of water. The medium was decanted and the aqueous phase was extracted with three times of 500 ml of diethyl ether. The organic phases were collected and washed three times with 500 ml of water and one time with 500 ml of a saturated aqueous NaCl solution in order to remove excess of dimethylaminoethanol. The organic phase was then dried over MgSO₄, filtered and evaporated to give 47.9 g of a crude dark oil. At this stage the crude contained a residual amount of the starting malonate.

The product was then purified by flash chromatography on silica gel with a first eluent consisting on CHCl₃/AcOEt mixture going through a gradient from 100% CHCl₃ to 100% AcOEt.

In order to remove all the product from the column, the column was also flushed with isopropanol+NEt₃ mixture (10% vol NEt₃) allowing getting additional pure product.

The clean fractions were collected affording, after solvent evaporation, 27.8 g of a pure product corresponding to 54% isolated yield.

NMR analysis showed that the product was in the form of a mixture of two position isomers with the following ratio: 54 mol % of the isomerized product (cis and trans diastereoisomers) and 46 mol % of methylenated product.

¹H NMR (CDCl₃, 400 MHZ) δ (ppm): 5.45-5.13 (m, 1H: isomer 2 cis+trans), 4.42 (s, 1H, isomer 2 cis or trans), 4.24-4.06 (m, 4H, isomer 1+2), 3.99 (s, 1H, isomer 2 cis or trans), 2.58-2.40 (m, 4H, isomer 1+2), 2.32-2.24 (m, 4H, isomer 1), 2.20 (s, 12H, isomer 1), 2.19 (s, 12H, isomer 2), 2.09-1.89 (m, 4H, isomer 2 cis+trans), 1.45-0.99 (m, 51 H, isomer 1+2), 0.81 (t, J=6.8Hz, 6H).

¹³C NMR (CDCl₃, 101 MHZ) δ (ppm): 168.60, 168.41, 165.49, 164.05, 132.07, 131.57, 131.12, 130.77, 123.73, 63.35, 62.76, 58.08, 57.49, 57.45, 53.45, 45.73, 34.45, 30.07, 30.03, 29.72, 29.68, 29.58, 29.53, 29.45, 29.38, 28.46, 28.43, 28.27, 28.09, 22.70, 14.13 (terminal CH₃).

### c) Methylation to Afford a Mixture of Compounds (X) and (XI)

All the reactions were conducted in carefully dried vessels and under an inert argon atmosphere.

Fresh commercial anhydrous THF and dimethylsulfate were used as such.

In a 1 L double-jacketed reactor equipped with a mechanical stirrer, a condenser, an addition funnel and a temperature probe were added 100 ml of dry THF and 6.9 mL of dimethylsulfate (9.14 g, 72 mmol, 2 eq.). A solution of 24.6 g of the esteramine (36 mmol, 1 eq.) in 154 mL of THF was preliminary prepared in the addition funnel and was progressively added into the reactor under stirring at room temperature in order to limit the temperature increase. The mixture was then stirred at room temperature under argon and the reaction progress was monitored by NMR analysis. After 2 hours the mixture was brought to 40° C. and 0.2 mL of dimethyl sulfate (2 mmol, 0.06 eq.) were added to allow stirring and to achieve complete conversion.

Reaction was completed after one hour of stirring at 40° C. and all the volatiles (THF and remaining DMS) were removed under vacuum in order to afford 33.15 g of a 95 mol % purity product as a beige wax QA₄ with 94% yield.

NMR analysis showed the presence of 2 position isomers with 55:45 ratio between isomerized derivative (cis and trans diastereoisomers) and conjugated non-isomerized methylenated derivative.

¹H NMR (MeOD, 400 MHZ) δ (ppm): 5.60-5.25 (m, 1H: isomer 2 cis+trans), 4.80 (s, 1H, isomer 2 cis or trans), 4.75-4.50 (m, 4H, isomer 1+2), 4.38 (s, 1H, isomer 2 cis or trans), 3.84-3.72 (m, 4H, isomer 1+2), 3.69 (s, 6H, isomer 1+2), 3.22 (s, 18H, isomer 2), 3.21 (s, 18H, isomer 1), 2.50-2.35 (m, 4H, isomer 1), 2.22-2.02 (m, 4H, isomer 2 cis+trans), 1.60-1.09 (m, 35 H, isomer 1+2), 0.90 (t, J=6.8 Hz , 6H).

¹³C NMR (MeOD, 101 MHz) δ (ppm): 169.22, 169.01, 168.96, 165.52, 134.16, 133.22, 132.94, 131.74, 65.90, 65.81, 60.23, 60.18, 59.73, 55.27, 54.66, 54.62, 35.66, 35.54, 33.24, 33.23, 31.76, 31.01, 30.94, 30.91, 30.87, 30.85, 30.77, 30.74, 30.71, 30.66, 30.65, 30.63, 30.60, 29.73, 29.62, 29.45, 29.27, 23.89, 14.61 (terminal CH₃).

### Part 4.C—Additional Mixtures of Monoquaternary Ammonium Compounds of Formula (I) With Diquaternary Ammonium Compounds

Eight additional surfactant materials are prepared by mixing various amounts of surfactant materials QA₁, QA₂, QA₃ and QA₄.

The weight percentages of monoquaternary ammonium compounds of formula (I) and of diquaternary ammonium compounds contained in surfactant materials QA₁, QA₂, QA₃ and QA₄ are compiled here below, the remaining wt. % corresponding to impurities:

TABLE 2

| | | Approximate | | |
|---|---|---|---|---|
| | | wt. % of | Approximate | Mono over |
| | Formula(e) of | mono- | wt. % of | diquaternary |
| | diquaternary | quaternary | diquaternary | ammonium |
| Surfactant | ammonium | ammonium | ammonium | compounds |
| material | compounds | compounds | compounds | ratio, in % |
| QA₁ | Formula (VI) | 65.2 | 27.6 | 70 |
| QA₂ | Formula (VI) | 85 | 10 | 90 |
| QA₃ | Formula (VI) | 0 | 95 | 0 |
| QA₄ | Formulae (X) | 0 | 94 | 0 |
| | and (XI) | | | |

The following mixtures QA₅ to QA₁₂ are prepared using convention mixing techniques by mixina QA₁ to QA₄ in appropriate proportions:

TABLE 3

| | | Mono-over di- |
|---|---|---|
| | | quaternary |
| | | ammonium |
| Additional | | compounds |
| surfactant | QA₁ to QA₄ | ratio, |
| materials | mixtures | in % |
| QA₅ | QA₁ + QA₂ | 80 |
| QA₆ | QA₁ + QA₃ | 50 |
| QA₇ | QA₁ + QA₃ | 30 |
| QA₈ | QA₁ + QA₃ | 10 |
| QA₉ | QA₂ + QA₄ | 80 |
| QA₁₀ | QA₂ + QA₄ | 60 |
| QA₁₁ | QA₂ + QA₄ | 40 |
| QA₁₂ | QA₂ + QA₄ | 20 |

Optionally, surfactant materials QA₁ to QA₁₂ are made available in the form of an aqueous or hydro-alcoholic solution.

### Example 5—Additional Biodegradability Tests

Biodegradability of surfactant materials QA₃ and QA₄ were measured according to the OECD standard 301.

The results of the biodegradability test are reported in Table 4.

TABLE 4

| Surfactant | |
|---|---|
| material | Biodegradability after 28 days |
| QA₃ | 92% (OECD 301F) |
| QA₄ | 17% (OECD 301D) |

The results show outstanding biodegradability for the glycine betaine bisester compound (surfactant material QA₃) and fair biodegradability for the mixture of diquaternary ammonium compounds of surfactant material QA₄. All this is achieved without detrimentally affecting the surfactant properties of the compounds.

### Example 6—Additional Evaluations of Adsorption Properties on Nanocellulose Crystals

Adsorption properties of surfactant materials QA₃ and QA₄ were measured in accordance with the protocol described in example 3.

Ranges of X and M of aggregation of CNC are summarized in Table 5. The lower range of aggregation X or M, the better the adsorption properties on negatively charged surface.

TABLE 5

| | Range of CNC | Range of CNC |
|---|---|---|
| | aggregation (Ratio) | aggregation |
| Surfactant | X = [surfactant]/[CNC] | (Mass fraction) |
| material | Xₘᵢₙ − Xₘₐₓ | Mₘᵢₙ − Mₘₐₓ |
| Fentacare ® TEP | 1-33 | 0.50-0.97 |
| Glycine betaine | <<Xₘᵢₙ − Xₘₐₓ range | <<Mₘᵢₙ − Mₘₐₓ range |
| diester QA₃ of | of Fentacare ® TEP, | of Fentacare ® TEP, |
| example 4-Part A | similar to QA₄ | similar to QA₄ |
| QA₄ (mixture of | 0.1-1.82 | 0.09-0.65 |
| compounds of | | |
| formulae (X) | | |
| and (XI)) of | | |
| example 4-Part B | | |

Fentacare® TEP was used as a comparison. Fentacare® TEP is a commercial surfactant representing the benchmark.

The data show that the surfactant properties of surfactant materials QA₃ and QA₄, which serve for the preparation of the mixtures QA₅ to QA₁₂ (in accordance with the present invention), are superior compared to the commercial surfactant Fentacare® TEP.

Overall, the compounds of formula (I) show a good combination of surfactant properties combined with a good biodegradabilty—a combination which is in many cases not achieved by commercial surfactants. Since the compounds of formula (I) are also easily available starting from internal ketones which are easily accessible from fatty acids or fatty acid derivatives, they also provide economical benefits over prior art ammonium surfactants.

The same attractive combination of surfactant and biodegradabilty properties is achieved with mixtures comprising comprising the compounds of formula (I) and the compounds of formula (VII). An additional advantage of such mixtures is that, by varying the respective proportions of the compounds of formulae (I) and (VII), it is possible to adjust the viscosity of the aqueous or hydro-alcoholic formulations prepared from the mixtures within a broad range of values, allowing for the use of such mixtures in a broad range of applications requiring different levels of viscosity.

1. An ionic mono-ammonium compound of formula (I) 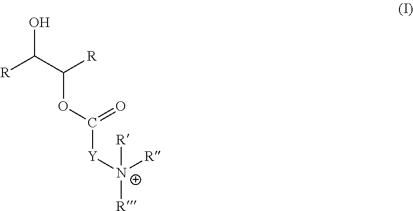
wherein
R, which may be the same or different at each occurrence, is a C₅-C₂₇ aliphatic group,
Y is a divalent C₁-C₆ aliphatic group, and
R′, R″ and R″′, which may be the same or different, are hydrogen or a C₁ to C₄ alkyl group.

2. The compound according to claim 1 wherein R is chosen from C₆-C₁₇ alkyl and C₆-C₁₇ alkenyl groups.

3. The compound according to claim 2 wherein R is a C₁₀-C₁₇ group.

4. The compound according to claim 1 wherein Y is a methylene group.

5. The compound according to claim 1 wherein R′, R″ and R″′ are methyl.

6. An electroneutral compound of formula (II) 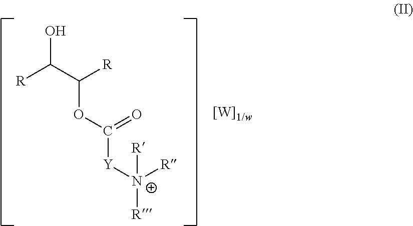
wherein
R is as defined in claim 1 for the compound of formula (I),
Y is as defined in claim 1 for the compound of formula (I),
R′, R″ and R″′ are as defined in claim 1 for the compound of formula (I), and
W is an anion or an anionic group bearing w negative charges.

7. The compound according to claim 6, wherein W is a halide anion and w is 1.

8. A monohydroxyl-monoester compound useful for the preparation of the compound of formula (I) of claim 1, said monohydroxyl-monoester compound being a compound of formula (III) 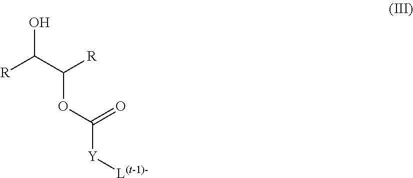
wherein
R, which may be the same or different at each occurrence, is as defined in claim 1 for the compound of formula (I),
Y, which may be the same or different at each occurrence, is as defined in claim 1 for the compound of formula (I),
L is a leaving group and
t is an integer which is at least 1.

9. The compound according to claim 8, wherein L is a nucleofuge group chosen from a halogen, a (hydrocarbyloxysulfonyl)oxy group of formula R^{a}—O—SO₂—O— wherein R^{a} denotes a C₁-C₂₀ hydrocarbyl group and an oxysulfonyloxy group of formula ⁻O—SO₂—O—.

10. A mixture M_{Q} comprising:
at least one ionic mono-ammonium compound of formula (I) as claimed in claim 1, and
at least one ionic di-ammonium compound of formula (VII) 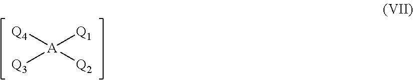
wherein
A is a tetravalent linker selected from the group consisting of A-1 to A-6 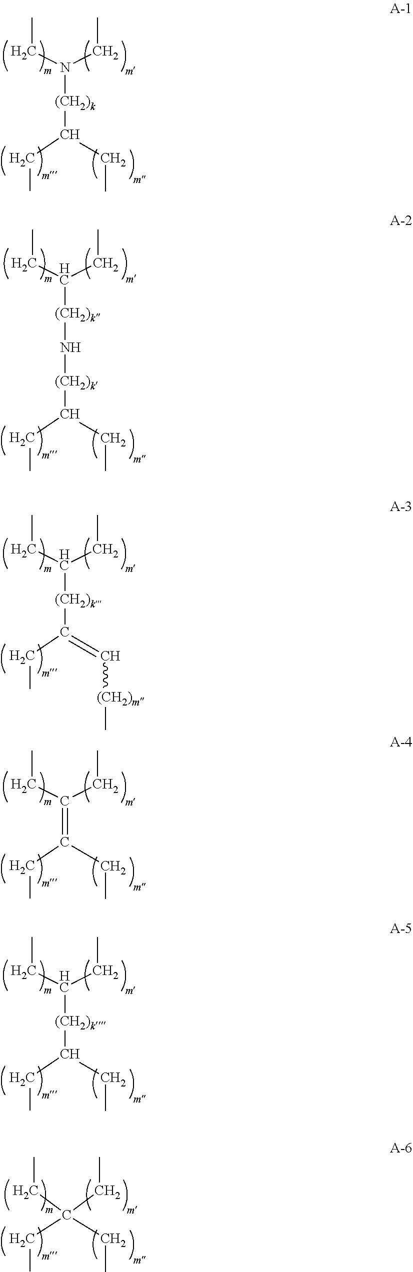
m, m′, m″ and m″′, which may be the same or different at each occurrence, are 0, 1, 2 or 3,
k, k′ k″, k″′ and k″′, which may be the same or different, are 0, 1, 2 or 3,
Q₁ to Q₄, which may be identical or different from each other, are selected from the group consisting of R and X,
R, which may be the same or different at each occurrence, is as defined in claim 1 for the compound of formula (I),
X, which may be the same or different at each occurrence, is represented by formula (VIII) 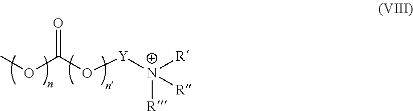
wherein
two and only two of Q₁ to Q₄ are represented by X and two and only two of groups Q₁ to Q₄ are represented by R,
Y is as defined in claim 1 for the compound of formula (I),
R′, R″ and R″′, which may be the same or different at each occurrence, are as defined in claim 1 for the compound of formula (I), and
n and n′, which may be the same or different at each occurrence, are 0 or 1 with the sum of n+n' being 1 or 2.

11. The mixture according to claim 10, wherein the ionic di-ammonium compound is of formula (VI) 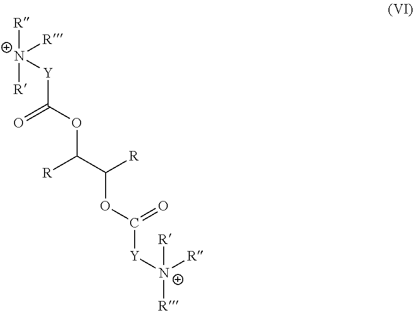
wherein
R, which may be the same or different at each occurrence, is as defined in claim 1 for the compound of formula (I),
Y, which may be the same or different at each occurrence, is as defined in claim 1 for the compound of formula (I), and
R′, R″ and R″′, which may be the same or different at each occurrence, are as defined in claim 1 for the compound of formula (I).

12. The mixture according to claim 10, wherein the ratio w_{I, VII} of the weight of the compound (I) over the combined weight of the compound (I) and the compound (VII) ranges from 10% to 90%.

13. A mixture M′_{Q} comprising:
at least one electroneutral compound of formula (II) according to claim 6 and
at least one electroneutral compound of formula (IX) 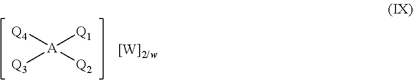
wherein A and Q₁ to Q₄, which may be identical or different from each other, are as defined for the ionic di-ammonium compound of formula (VII) comprised in the mixture M_{Q} according to claim 10,
and
W is an anion or an anionic group bearing w negative charges.

14. The mixture according to claim 13, wherein the ratio wILIx of the weight of the compound (II) over the combined weight of the compound (II) and the compound (IX) ranges from 10% to 90%.

15. (canceled)

16. The M′_{Q} of claim 13 wherein 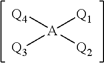
is the ionic di-ammonium compound of formula (VI) comprised in the mixture M_{Q} according to claim 11, and
W is an anion or an anionic group bearing w negative charges.

17. The mixture M′_{Q} of claim 13, wherein W is a halide anion and w is 1.

18. The mixture according to claim 10, wherein the ratio wIvu of the weight of the compound (I) over the combined weight of the compound (I) and the compound (VII) ranges from 50% to 90%.

19. The mixture according to claim 13, wherein the ratio worx of the weight of the compound (II) over the combined weight of the compound (II) and the compound (IX) ranges from 50% to 90%.
